(19) United States
(12) Patent Application Publication
(10) Pub. No.: US 2025/0266121 A1
(43) Pub. Date: Aug. 21, 2025

(54) A METHOD FOR PROTEIN DESIGN

(71) Applicant: European Molecular Biology Laboratory, Heidelberg (DE)

(72) Inventors: Michael JENDRUSCH, Heidelberg (DE); Jan KORBEL, Heidelberg (DE); Kashif SADIQ, Heidelberg (DE)

(73) Assignee: European Molecular Biology Laboratory, Heidelberg (DE)

(21) Appl. No.: 18/856,256

(22) PCT Filed: Apr. 11, 2023
(86) PCT No.: PCT/EP2023/059466

§ 371 Date: Oct. 11, 2024

Related U.S. Application Data
(60) Provisional application No. 63/329,522, filed on Apr. 11, 2022.

(51) Int. Cl. G16B 15/20; G16B 15/30; G16B 30/00; G16B 40/20

(52) U.S. Cl. CPC G16B 15/20; G16B 15/30; G16B 30/00; G16B 40/20

(57) ABSTRACT

Provided is a computer implemented method for designing at least one protein includes creating at least one amino acid sequence to be tested, wherein ones of the amino acids, contained in the amino acid sequence to be tested, are selected according to a probability distribution; predicting, from the aligned at least one amino acid sequence, structural properties of the at least one protein; calculating, based on the structural properties of the at least one protein, a value of a fitness function for the at least one amino acid sequence to be tested; selecting or deselecting, dependent on the value of the fitness function, the at least one amino acid sequence to be tested. The amino acid sequence may be post-processed to be more native-like, have enhanced solubility, and/or improved expression.

### CROSS-REFERENCE TO OTHER APPLICATIONS

This application claims priority to U.S. provisional application No. 63/329,522, filed on 11 Apr. 2022, the disclosure of which is hereby incorporated herein by reference in its entirety. This application claims priority to international patent application no. PCT/EP/2023/059466, filed on 11 Apr. 2023, the disclosure of which is hereby incorporated herein by reference in its entirety.

### BACKGROUND OF THE INVENTION

### Field of the Invention

The invention relates to a method and an apparatus for the design of proteins.

### Brief Description of the Related Art

De novo protein design is a longstanding fundamental goal of synthetic biology but has been hindered by the difficulty in reliable prediction of accurate high-resolution protein structures from sequences of amino acids. Recent advances in the accuracy of protein structure prediction methods, such as AlphaFold (AF), have facilitated proteome scale structural predictions of monomeric proteins.

Proteins are the workhorses of most life processes at the cellular scale. Proteins enable a myriad of functions ranging from the catalysis of biochemical reactions, mechanical functions involved in cell motility and the formation of sub-cellular architecture amongst many others. A central paradigm to understand function of the proteins has been based on the observation that proteins fold into complex, yet specific three-dimensional structures that vary depending on their amino acid sequence, hypothesized to be their lowest energy state. Thus, experimental structure determination has been a primary pursuit of biology for the last 50 years and given rise to over 10⁵ distinctly solved structures, deposited in the protein data bank (PDB) [1]. These structures have revealed a diverse array of fold topologies and geometries of individual proteins, classified into 41 architectures with 1390 fold topologies and that 51% of structures previously extracted from the PDB form quaternary structure through oligomer and complex formation.

A question has been to what extent the amino acid sequence encodes the protein structures and whether it is then possible to predict the protein structure from the amino acid sequence. This question has fueled a plethora of diverse computational protein structure prediction approaches across decades of effort [2] The quest for improved methods has been embodied in an independently assessed biennial community-wide competition (CASP) that ranks method accuracy of participants' approaches with respect to determined but unreleased structures resulting in steady progress towards predictive accuracy [3]. This question has recently culminated in the development of AlphaFold [4]—hereafter, termed AF. AF is a neural network-based approach that has achieved comparable atomic accuracy with respect to crystal structure. AF has been applied at a proteome-wide scale across several species, resulting in a new database of predicted structures [5]. More recently, a community-wide assessment has revealed several important applications of AF ranging from amino acid variant effect prediction to cryo-EM model building [6]. Whilst intended for single-chain structure prediction, an unexpected consequence of AF's input protocol allows prediction of non-contiguous chains, thus enabling prediction of protein complexes using the existing trained network [7]. This feature has also been applied at proteome-wide scale to reveal novel core eukaryotic complexes [8]. These efforts culminated in the release of AlphaFold-Multimer [9], a version of AF explicitly trained for complex prediction.

Despite these advances, the protein folding problem remains far more complex than structure prediction alone. Proteins are not rigid structures, for example at physiological conditions. The proteins exhibit thermodynamic equilibrium between folded and unfolded forms. Many proteins also undergo conformational changes with a well-described equilibrium between their states, making use of these changes to enact function [10]. Moreover, there is an abundance of intrinsically disordered proteins (IDPs)—those that do not exhibit stable tertiary structures, or transiently fold depending on environmental context [10]. One notable biomedical example being the misfolding of amyloid-α helices into β-sheeted filaments associated with Alzheimer's disease [11]. Metamorphic proteins have also been discovered. These metamorphic proteins form multiple, stable, yet different folds [12]

From a biophysical perspective, folding of the proteins is made comprehensible using the concept of a folding energy funnel within the atomic configuration space. Multiple energy minima then correspond to alternate stable states for the proteins that make transitions with characterizable rates. Based on this concept, computational physics methods such as molecular dynamics (MD) simulations have provided a route to characterize the dynamics, thermodynamics, and kinetics of conformational transitions [13], ab initio folding disordered transitions as well as protein-protein and protein-ligand [14] binding.

Notwithstanding the already-mentioned complex structure and dynamics of naturally occurring proteins, evolution has still only explored an infinitesimal portion of the potential protein sequence landscape [15]. There is therefore enormous potential in unlocking the fundamental biophysical principles of protein folding to design and engineer novel proteins that can exploit this vast space. Recent examples include protein logic gates [16], self-assembling systems [17] and targeted therapeutics [18].

Established methods for protein engineering have until recently focused on tuning naturally occurring proteins through iterative experimental selection processes such as directed evolution [19]. More recently, computational design approaches have enabled de novo protein design, encompassing a full suite of functionalities ranging from rules for topology selection, protein backbone construction, optimization of the sequences of amino acids, as well combinations of these approaches [20].

One prior art method in computational protein design is embodied by the Rosetta suite of protein design tools [21] and Rosetta Remodel [22] in particular. The Rosetta suit combines tools for all steps from selecting a desired protein topology to designing and validating a folding protein sequence of amino acids. The design of the amino acid sequences for the proteins within Rosetta Remodel is composed of a combination of three tasks: topology specification, backbone generation and fixed-backbone design [22]. After specifying a desired protein topology, a backbone structure for the protein can be generated using matching fragments extracted from existing proteins. Fragments matching a desired secondary structure and set of contacts are selected from PDB structures. These selected fragments are then sampled using Markov-Chain Monte Carlo to arrive at a pool of candidate backbone geometries. The candidate backbone structures can then be equipped with a sequence using fixed-backbone protein design and, optionally, optimizing the backbone between design steps [23]. Given a desired backbone geometry, the goal is to find an amino acid sequence which will fold that protein structure. Here, the Rosetta Design protocol [23] starts by populating the desired backbone with an all-valine sequence and runs Markov-Chain Monte Carlo to arrive at a low-energy sequence. The general strategy of the Rosetta-based protein design has been successfully applied to a variety of design problems. These applications range from the first de novo designed proteins [23] to synthetic vaccines [24], complex assemblies [25] and enzymes [26]. However, the Markov-Chain Monte Carlo in structure space can be time-consuming and computationally demanding.

To tackle this shortcoming of classical protein design, there have been approaches applying neural networks to various design problems. Several works train generative models to directly generate protein sequences with a desired function. For example, [27] have trained a language model on the UniProt sequence database to generate sequences with a specified function and fine-tuning the generative model on a specific protein family or function. Beyond language models, other types of generative models, such as variational autoencoders [28] and generative adversarial networks [29] have been explored for direct protein sequence generation. While these approaches have been successful in generating protein sequences associated with a given function, the approaches do not explicitly consider structural information and thus cannot be applied to protein design tasks involving constraints on tertiary structure.

On the other side of the spectrum of neural network-based protein design, generative models have been explored for protein structure generation [30] have trained generative adversarial networks to generate realistic backbone distance maps and coordinates. [31] have achieved the same goal using variational autoencoders. Both approaches have demonstrated fine-grained control over designed backbone structures by latent variable manipulation. These approaches are a viable alternative to classical backbone design, but the approaches offer no guarantees on the designability of their predicted backbones.

Bridging the gap between the structure-only and the sequence-only approaches, [32] have trained the neural networks on the PDB database of protein structures to predict the protein sequence given a fixed backbone structure. These approaches rely on network components taking into account the geometry of the protein backbone together with the protein sequence, including the use of per-residue local coordinate systems to reason about protein geometry.[33] have framed fixed-backbone design as a constraint satisfaction problem and have trained their network as a constraint solver. These methods require a neural network trained for a specific protein design task—fixed-backbone protein design—and by themselves cannot easily be extended to other design applications without retraining.

Prior art has also explored re-using previously trained predictors of protein structure or function as parts of an in silico screening framework. In these approaches, a neural network is treated as a score function to evaluate the quality of protein designs [34]. Designs are then improved using gradient-based [35], gradient-free [36] or neural-network based [37] optimization.

[34] were the first to use an optimization loop incorporating a neural network for structure prediction for de novo protein design. Their approach has since been extended to fixed-backbone design [35] and protein scaffold generation for protein motif stabilization, using both Markov-Chain Monte Carlo and gradient descent as their meansfor optimization. All these approaches have made use of trRosetta [38] as a structure predictor. [36] have used a new release of AF as a structure predictor for fixed-backbone protein design using greedy optimization, with sequences initialized from a trained model.

### SUMMARY OF THE INVENTION

In a preferred embodiment the present invention is a computer implemented method for protein design.

A computer implemented method for designing at least one protein comprises creating at least one amino acid sequence to be tested, wherein ones of the amino acids, contained in the amino acid sequence to be tested, are selected according to a probability distribution; predicting, from the aligned at least one amino acid sequence, structural properties of the at least one protein; calculating, based on the structural properties of the at least one protein, a value of a fitness function for the at least one amino acid sequence to be tested; selecting or deselecting, dependent on the value of the fitness function, the at least one amino acid sequence to be tested.

The method may further comprises altering the at least one amino acid sequence to be tested, by changing at least one of the amino acids contained in the amino acid sequence to be tested.

The method may further comprise repeating the altering of the at least one amino acid sequence until the value of the fitness function exceeds a pre-selected threshold.

The predicting of the structural properties of the least one protein, contained in the amino acid sequence to be tested, may comprise predicting locations of the amino acids with respect to each other and/or predicting an all-atom structure of the at least one protein.

The method may use amino acid sequence (also termed protein sequence) optimization with gradient-free or gradient-based optimizers. An example of a gradient-free optimizer is an evolutionary algorithm. The optimization produces backbones (i.e., peptide-bond-linked amino acids or polypeptides, from which sidechains branch off) and amino acid sequences which are, by construction, designable and with high confidence under AF. Using the evolutionary algorithm enables automation of searching the amino acid sequences (also termed protein sequences). The altering may comprise mutating and/or recombining the at least one amino acid sequence to be tested, according to an evolutionary algorithm.

The structural properties may be predicted using AlphaFold. AlphaFold (AF) [4] may be embedded into a design loop for predicting protein structures. By searching for the amino acid sequences with high-confidence predictions, for example, native-like protein structures can be predicted, e.g., by means of AF. A flexible family of fitness functions encoding various protein design tasks is designed. This family of fitness functions is integrated into the design loop with extensive validation using Rosetta [21] and/or molecular dynamics simulations.

The fitness function may be defined according to biological and/or physicochemical properties of the at least one protein.

The fitness function comprises one or more fitness function components, which represent the biological and/or physicochemical properties of the at least one protein.

The method may further comprise inputting known structural properties associated with the at least one amino acid sequence or a target protein, to which the at least one protein is bindable, into AlphaFold as a structural template.

The method enables rapid prediction of novel protein monomers starting from random sequences. These novel protein monomers can adopt a diverse array of folds within the known protein space. The method can be used for designing proteins that bind to a pre-specified target protein.

It has been found that the protein monomers demonstrate widespread maintenance of structural integrity. The method also reveals the capacity to predict proteins that switch conformation upon complex formation, such as involving switches from α-helices to β-sheets during amyloid filament formation.

The method may further comprise re-designing the selected at least one amino acid sequence to be tested, to make the re-designed at least one amino acid sequence more native-like and/or to improve a solubility and/or an expressability of the at least one protein.

The method may further comprise statistically recovering the at least one amino acid sequence to be tested, from the predicted structural properties of the least one protein. From the predicted protein structure, a recovered amino acid sequence may thus be calculated.

The calculating of the recovered amino acid sequence enables further validation of the method of the present disclosure.

The method may further comprise re-predicting structural properties of the at least one protein based on the re-designed at least one amino acid sequence. From the recovered amino acid sequence, a re-predicted protein sequence may be computed. The calculating of the re-predicted protein structure enables further validation of the method of the present disclosure.

The method may further comprise computationally validating the selected amino acid sequence using molecular dynamics and/or Rosetta ab-initio structure prediction.

The structural integrity of the predicted protein structures has been validated and confirmed by standard ab initio folding and structural analysis methods as well as more extensively by performing rigorous all-atom molecular dynamics simulations and analyzing the corresponding structural flexibility, intramonomer and interfacial amino-acid contacts.

The method may further comprise experimentally validating the selected amino acid sequence by determining a solubility and/or an expressibility of the at least one protein.

The at least one protein may comprise a monomer, a binder binding to a target protein, a homodimer, a heterodimer, a trimer, a tetramer, a pentamer, an oligomer, or a protein complex.

### DESCRIPTION OF THE FIGURES

For a more complete understanding of the present invention and the advantages thereof, reference is now made to the following description and the accompanying drawings, in which:

FIG. 1 shows a method overview. (A) The method according to the disclosure includes defining a fitness function; generating structures (candidate structures) by generating candidate amino acid sequences using evolutionary search; re-designing the amino acid sequences to be more native-like; re-predicting structures of the re-designed amino acid sequences; filtering of the re-designed amino acid sequences by likelihood, solubility and prediction confidence; codon-optimizing remaining amino acid sequences; ordering codon-optimized amino acid sequences for down-stream experiments. (B) The method includes an optimization loop. A pool of amino acid sequences (sequence pool) is maintained throughout the design process. AlphaFold predicts the all-atom structure (all-atom geometry) and confidence metrics (pLDDT, pAC) for each sequence. These are combined into a fitness function that is optimized, e.g., maximized or minimized, in the optimization loop. The sequences of the amino acids (also termed protein sequences below) and fitness values of the fitness function, associated with the sequences of the amino acids, are fed to an optimizer, which updates the sequence pool, e.g., by means of an evolutionary algorithm. If the fitness value, associated with a selected one of the sequences of amino acids, exceeds a desired threshold, the sequence of amino acids and structure are submitted for validation using Rosetta and molecular dynamics simulations. The fitness function may include one or more fitness function components. The fitness function may be any function of the amino acid sequence, the predicted all-atom structure and the confidence metrics (pLDDT, pAC). Examples of the fitness function include a root mean square deviation (RMSD) with respect to a reference structure (e.g., a native structure, an experimentally validated structure, a decoy structure, or the like), one or more of the confidence metrics, or constraints on complex formation and/or conformational change upon complex formation. These and further examples of fitness function components used to construct the fitness function. and mathematical expressions of these fitness function components are described in detail below.

FIG. 2 shows AlphaFold prediction of protein complexes. AlphaFold predictions of dimers (HIV protease 1DMP, de novo designed protein 4PWW), trimers (de novo designed coiled coil 1COI), tetramers (consensus TPR superhelix 2AVP), pentamers (phage lambda tail terminator 3FZB). Predictions (dark grey) are aligned and overlaid onto the native structure (light grey). All predictions show low RMSD to the native structure.

FIG. 3 shows de novo monomer design. (A-D) ab initio and molecular dynamics validation of designed monomers of length 32-256 amino acids. Designed monomers (light grey) are overlaid with their lowest-energy structure (dark grey) from Rosetta ab initio prediction or relaxation of their predicted all-atom structure (structures). Each monomer is reported with its RMSD with respect to the predicted AlphaFold structure and its value, also termed “Rosetta Score”, of a Rosetta All-atom Energy Function, e.g., REF2015 or an energy function based on electrostatic interactions, van-der-Waals interactions, and/or statistical physics. Predicted aligned confidence (pAC) is shown for each designed monomer (predicted error). For validation using Rosetta, a distribution of Rosetta scores and RMSDs to the AlphaFold structure is shown (Rosetta score distribution): for the top ten relaxed structures (relaxed) starting from ab initio prediction (dark grey) and the AlphaFold structure (light grey); for all decoys (i.e., conformations having a low free energy) from Rosetta ab initio prediction (ab initio) starting from an extended conformation (light grey) and the AlphaFold structure (dark grey). For molecular dynamics validation (MD metrics), the Boltzmann-weighted collective variable (CV) distribution in the 2D landscape of the number of intramonomer amino acid contacts and backbone RMSF (root mean square fluctuation) of the protein with respect to the averaged MD structure is shown (CV distribution). (E) TSNE (t-distributed stochastic neighbor embedding) of designed monomer structures of length 64 amino acids and larger. Structures are separated into ten clusters using agglomerative clustering. (F) Representatives for each cluster, showing diverse structures designed using AlphaFold.

FIG. 4 shows, for de novo dimer design, (A-C) Rosetta and molecular dynamics validation of designed homodimers of length 32-128 amino acids. Designed homodimers are overlaid with their lowest-energy structure resulting from Rosetta relaxation of their predicted all-atom structure (complex structures). Each relaxed dimer is reported with its RMSD to the AlphaFold structure, Rosetta binding energy and packing statistics. Predicted aligned confidence (pAC) is shown for each designed dimer (predicted error). For molecular dynamics validation (MD complex metrics), the Boltzmann-weighted CV distribution in the 2D space of total amino acid contacts (intramonomer and interfacial) and RMSF of the complex is shown (CV distribution), together with the distribution of individual monomer contacts and interface contacts. (D-F) Rosetta and molecular dynamics validation of designed heterodimers of length 32-128 amino acids. Measures displayed are the same as in (A-C). (G) Orthogonality of designed dimers. For two distinct pairs of designed dimers, predicted structures for the on-target and off-target complexes are shown. Predicted confidence (predicted error) of the on-target dimers is consistently higher for inter-monomer pairs of amino acids than the same confidence for off-target dimers.

FIG. 5 shows de novo oligomer design. (A-G) Rosetta and molecular dynamics validation of designed trimers, tetramers and pentamers of monomer length 32-64 amino acids, as well as hexamers of monomer length 32 amino acids. Designed oligomers are overlaid with their lowest-energy structure from Rosetta relaxation of their predicted all-atom structure (complex structures). Each relaxed oligomer is reported with its RMSD with respect to the AlphaFold structure, Rosetta binding energy and packing statistics. Predicted aligned confidence (pAC) is shown for each designed oligomer (predicted error). For molecular dynamics validation, the Boltzmann-weighted CV distribution in the 2D space of total amino acid contacts (intramonomer and interfacial) and RMSF of the complex is shown (CV distribution), together with the distribution of individual monomer contacts and interface contacts. (H) Additional structures.

FIG. 6 shows de novo binder design. (A, B) Rosetta validation of designed binders for previously de novo designed proteins. Relaxed binders bound to the target protein are overlaid with their predicted all-atom structure (complex structures). Each relaxed binder is reported with its RMSD with respect to the AlphaFold structure, Rosetta binding energy and packing statistics. Predicted aligned confidence (pAC) is shown for each designed binder (predicted error). For molecular dynamics validation, the Boltzmann-weighted CV distribution in the 2D space of total amino acid contacts (intramonomer and interfacial) and RMSF of the complex is shown (CV distribution), together with the distribution of individual monomer contacts and interface contacts.

FIG. 7 shows Conformational change in AlphaFold sequence space and de novo designed proteins. (A) AlphaFold prediction of conformational change upon complex formation. (left) Predicted structures for monomeric circadian clock protein KaiB (KaiB ground state, KaiBgs) and fold-switch stabilized KaiB (KaiBfs) predicted in complex with KaiC (dark grey) overlaid with the native structure of KaiBgs (1R5P) and KaiBfs (5JYT), respectively. Both predictions show low RMSD with respect to the corresponding native structure. Predicted KaiBfs (dark grey) overlaid with native KaiBgs (the incorrect conformation, light grey) shows high RMSD. (right) Predicted structures for monomeric Amyloid-β and its pentamer (dark grey) overlaid with the native monomer (IIYT) and oligomer (2MXU). AlphaFold predicts the conformational change from α-helix to parallel β-sheet characteristic for amyloids. (B) Rosetta and molecular dynamics validation of designed oligomers of monomer length 32 showing amyloid-like predicted conformational change. Designed oligomers (light grey) are overlaid with their lowest-energy structure (dark grey) from Rosetta relaxation of their predicted all-atom structure (complex structures). Each relaxed oligomer is reported with its RMSD to the AlphaFold structure, Rosetta binding energy and packing statistics. Monomers are shown in comparison to the oligomer structure (monomer structures) to illustrate conformational change upon oligomerization. Predicted aligned confidence (pAC) is shown for each designed oligomer and its constituent monomers (predicted error). For molecular dynamics validation, the Boltzmann-weighted CV distribution in the 2D space of total amino acid contacts (intramonomer and interfacial) and RMSF of the complex is shown (CV distribution), together with the distribution of individual monomer contacts and interface contacts. (C) Additional proteins designed using a fitness function favoring conformational change upon complex formation.

FIG. 8 shows designed monomer validation using Rosetta and molecular dynamics. (A-D) Validation of de novo designed monomers of length 32, 64, 128 and 256 amino acids using Rosetta and molecular dynamics. (structures). The lowest Rosetta energy structure is shown overlaid with the predicted AlphaFold structure, reporting RMSD and Rosetta all-atom score. (predicted error) shows the predicted aligned confidence for each monomer. (Rosetta score distribution) shows the distribution of decoys (i.e., conformations having a low free energy) from relaxation and ab initio prediction over Rosetta energy and RMSD to the predicted AlphaFold structure. (relaxed) shows this distribution for relaxations of the ten lowest-energy ab initio decoys (dark grey) and ten relaxations of the AlphaFold predicted structure (light grey). (ab initio) shows the distribution of decoys with Rosetta energy<OREU sampled starting from an extended conformation (grey) and the AlphaFold structure (red). For molecular dynamics-based validation, (RMSD/F evolution) shows the time evolution of RMSD (dark grey) and RMSF (light grey) for each monomer over the course of 100 ns of explicit-solvent molecular dynamics simulation. (contact evolution) shows the time evolution of the number of intra-monomer contacts over 100 ns of simulation (grey). (CV distribution) shows the Boltzmann-weighted distribution in the 2D collective variable (CV) landscape of backbone RMSF and total contacts (intramonomer and interfacial) for all snapshots in the last 50 ns of simulation. E additional random designed structures for monomers of length 64 and 128 amino acids.

FIG. 9 shows designed dimer validation using Rosetta and molecular dynamics. (A-F) Validation of de novo designed homo and heterodimers of monomer length 32, 64 and 128 amino acids using Rosetta and molecular dynamics. (complex structures) shows the AlphaFold predicted structure (light grey) overlaid with the best of 10 relaxations (dark grey) using the Rosetta all-atom score function. Structures are reported with their RMSD to the relaxed structure, as well as Rosetta binding energy dG and packing statistics packstat. (monomer structures) shows the AlphaFold predicted structure of the monomer (dark grey) overlaid with the structure of the complex (light grey) and reports aligned RMSD. (predicted error) shows the predicted aligned confidence for each AlphaFold complex prediction. For molecular dynamics-based validation, (RMSD/F evolution) shows the time evolution of RMSD (dark grey) and RMSF (light grey) of the entire complex over the course of 100 ns of explicit-solvent molecular dynamics simulation. (contact evolution) shows the time evolution of total intra-monomer contacts as well as interface contacts over the course of 100 ns of simulation. (CV distribution) shows the Boltzmann-weighted distribution in the 2D collective variable (CV) landscape of backbone RMSF and total contacts (intramonomer and interfacial) for the entire complex for all snapshots in the last 50 ns of simulation. (MD monomer metrics) reports the distributions of RMSD (monomer RMSD), RMSF (monomer RMSF), monomer contacts and interface contacts for all monomers and interfaces in the complex over the last 50 ns of simulation.

FIG. 10 shows Designed oligomer validation using Rosetta and molecular dynamics. (A-D) Validation of de novo designed trimers, tetramers, pentamers and hexamers of monomer length 32 and 64 amino acids using Rosetta and molecular dynamics. (complex structures) shows the AlphaFold predicted structure (light grey) overlaid with the best of 10 relaxations (dark grey) using the Rosetta all-atom score function. Structures are reported with their RMSD to the relaxed structure, as well as Rosetta binding energy of a single monomer to the rest of the complex dG and packing statistics packstat. (monomer structures) shows the AlphaFold predicted structure of the monomer (dark grey) overlaid with the structure of the complex (light grey) and reports aligned RMSD. (predicted error) shows the predicted aligned confidence for each AlphaFold complex prediction. For molecular dynamics-based validation, (RMSD/F evolution) shows the time evolution of RMSD (dark grey) and RMSF (light grey) of the entire complex over the course of 100 ns of explicit-solvent molecular dynamics simulation. (contact evolution) shows the time evolution of total intra-monomer contacts as well as interface contacts over the course of 100 ns of simulation. (CV distribution) shows the Boltzmann-weighted distribution in the 2D collective variable (CV) landscape of backbone RMSF and total contacts (intramonomer and interfacial) for the entire complex for all snapshots in the last 50 ns of simulation. (MD monomer metrics) reports the distributions of RMSD (monomer RMSD, blue), RMSF (monomer RMSF, green), monomer contacts (grey) and interface contacts (orange) for all monomers and interfaces in the complex over the last 50 ns of simulation.

FIG. 11 shows designed binding protein validation using Rosetta and molecular dynamics. (A, B) Validation of de novo designed binders of length 64 amino acids for two previously designed target proteins using Rosetta and molecular dynamics. (target protein) shows the AlphaFold predicted structure for the target protein. (complex structures) shows the AlphaFold predicted structure of the target protein in complex with the binder (grey) overlaid with the best of 10 relaxations (colored) using the Rosetta all-atom score function. The target protein is colored blue, all designed binders are colored red. Structures are reported with their RMSD to the relaxed structure, as well as Rosetta binding energy dG and packing statistics packstat. (monomer structures) shows the AlphaFold predicted structure of the monomer (dark grey) overlaid with the structure of the complex (light grey) and reports aligned RMSD. (predicted error) shows the predicted aligned confidence for each AlphaFold complex prediction. For molecular dynamics-based validation, (RMSD/F evolution) shows the time evolution of RMSD (dark grey) and RMSF (light grey) of the entire complex over the course of 100 ns of explicit-solvent molecular dynamics simulation. (contact evolution) shows the time evolution of total intra-monomer contacts as well as interface contacts over the course of 100 ns of simulation. (CV distribution) shows the Boltzmann-weighted distribution in the 2D collective variable (CV) landscape of backbone RMSF and total contacts (intramonomer and interfacial) for the entire complex for all snapshots in the last 50 ns of simulation. (MD monomer metrics) reports the distributions of RMSD (monomer RMSD), RMSF (monomer RMSF), monomer contacts and interface contacts for all monomers and interfaces in the complex over the last 50 ns of simulation.

FIG. 12 shows Designed conformation-changing oligomer validation using Rosetta and molecular dynamics. (A-D) Validation of de novo designed trimers, tetramers and pentamers of monomer length 32 amino acids using Rosetta and molecular dynamics. (complex structures) shows the AlphaFold predicted structure (light grey) overlaid with the best of 10 relaxations (dark colored) using the Rosetta all-atom score function. Structures are reported with their RMSD to the relaxed structure, as well as Rosetta binding energy of a single monomer to the rest of the complex dG and packing statistics packstat. (monomer structures) shows the AlphaFold predicted structure of the monomer (dark grey) overlaid with the structure of the complex (light grey) and reports aligned RMSD. (predicted error) shows the predicted aligned confidence for each monomer (left) and complex (right) AlphaFold prediction. For molecular dynamics-based validation, (RMSD/F evolution) shows the time evolution of RMSD (dark grey) and RMSF (light grey) of each complex over the course of 100 ns of explicit-solvent molecular dynamics simulation. (contact evolution) shows the time evolution of total intra-monomer contacts as well as interface contacts over the course of 100 ns of simulation. (CV distribution) shows the Boltzmann-weighted distribution in the 2D collective variable (CV) landscape of backbone RMSF and total contacts (intramonomer and interfacial) for the entire complex for all snapshots in the last 50 ns of simulation. (MD monomer metrics) reports the distributions of RMSD (monomer RMSD), RMSF (monomer RMSF), monomer contacts and interface contacts for all monomers and interfaces in the complex over the last 50 ns of simulation.

FIG. 13 shows prediction runs.

FIG. 14 shows (A) snapshots of a monomer at increasing fitness function values (pAC) during the optimization. For increasing fitness function values (pAC) from 0.7 to 1.5, the predicted structure (top) changes. Simultaneously, local confidence for each amino acid (pLDDT, top) and predicted aligned confidence for each pair of amino acids (pAC, bottom) increase. (B) Snapshots of a heterodimer at increasing fitness function values (pAC) during optimization are shown. For increasing fitness function values from 0.8 to 1.5, the structure of the monomers changes and the monomers are predicted to be in closer proximity. In parallel, the per-monomer and interface predicted aligned confidence approach 1.0, indicating increasing confidence with which the designed monomers form a heterodimer. (C) Fitness function components involved in monomer and dimer design are schematically shown. For monomer design, predicted LDDT is maximized at each sequence position, pAC is maximized for each pair of amino acids and the mean and maximum protein radius is constrained to ensure compactness. For dimer and oligomer design, pAC is additionally maximized for both the constituent monomers and the whole complex.

FIG. 15 shows results for a toxin inhibition conjugation (TIC) screening of blockers designed in silico according to the present disclosure. (A) A TIC screening against RcaT-Sen2 using IPTG-inducible high-copy plasmids which express the 88 designed blockers (donor-library). An arrayed donor-library (384-density format) was conjugated with an E. coli BW25113 recipient which carries an arabinose-inducible RcaT-Sen2 plasmid. These double-plasmid-bearing transconjugants (i.e., the afore-mentioned E. coli recipient having one of the IPTG-inducible plasmids as well as the arabinose-inducible plasmid) were selected and colony opacity was extracted using Iris tool [58] for assessing fitness of the transconjugants. For each transconjugant strain, opacity was normalized against the mean opacity of GFP-expressing negative controls in each plate (which do not affect toxin activity). The mean blocking score (see x-axis) was calculated by dividing the mean normalized opacity of each strain in experimental plates (n=2 biological) containing Arabinose+IPTG (RcaT and blocker induced) with the mean normalized opacity of control plates containing only IPTG (blocker induced) (n=2 biological). Mean blocking score with SD (cut-off>1.5), neutral scores are shown in grey while hits in black are called for p<0.01 following t-test comparing mean blocking score of experimental plate and control plate. (B). Analogous results as in (A), but with recipient E. coli BW25113 carrying the RcaT-Eco9 plasmid.

### DETAILED DESCRIPTION OF THE INVENTION

The present disclosure relates to an illumination unit for use in optical applications. One example of the optical applications is microscopy. In particular, the illumination unit is useful for variants of microscopy, in which a patterned illumination light beam 12 with an intensity minimum (also termed “zero”) is scanned around a sample located at a sample position. One example of such variant of microscopy is the so-called MINFLUX microscopy.

The method of this document is a search problem to find a set of sequences of amino acids for a protein (amino acid sequences) for which a mathematical fitness function exceeds a fixed threshold to fit a design goal for the protein. The mathematical function is a combination of the sequence of amino acids forming the protein, a predicted structure for the protein as well as AF confidence measures, such as pAE and pLDDT (explained later). The design goal for the protein could be simply proper protein folding or could be some biological function, such as binding to a target protein, dimerization, self-assembly, etc. The method uses AF to take into account both properties of the protein sequence of the amino acids (also termed “amino acid sequence”) as well as all-atom protein structure of the protein sequences of the amino acids, which are integrated into the fitness functions, to provide the structure prediction of the proteins as well as the measures of prediction confidence.

The method of this document is combined with validation using Rosetta ab initio structure prediction [21] and molecular dynamics simulations.

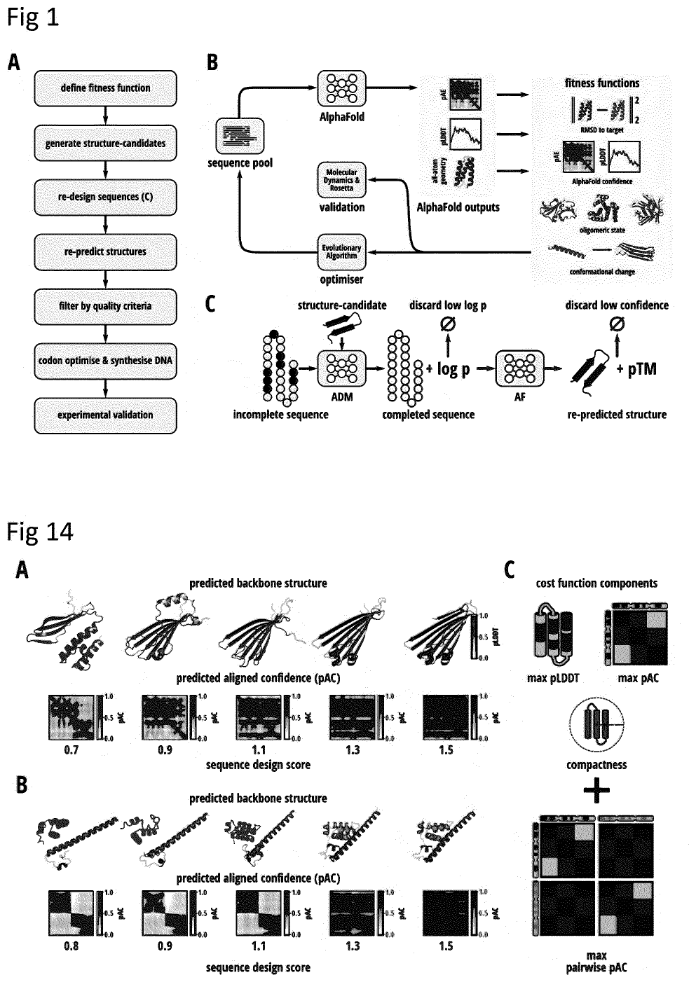

FIG. 1 shows an outline of the method. An optimization loop is set up which iteratively mutates a sequence pool of sequences of the amino acids, scores the sequence pool of the amino acid sequences using the output of AF to model the fitness function and subsequently updates the sequence pool with the mutated sequences of the amino acids and scores, as is indicated (see FIG. 1A). An initial pool of the amino acid sequences (or protein sequences of amino acids) is created by sampling, in one aspect, sequences of amino acids of a fixed length uniformly at random from the 20 standard amino acids. In another aspect, it is possible to exclude ones of the standard amino acids, such as cysteine, from the creating of the initial pool of the amino acid sequences to avoid designs of the proteins containing disulfide bonds. Alternatively, the initial pool may be initialized or created by sampling from a pre-defined probability distribution of the amino acids or from a pre-defined probability distribution of the amino acid sequences, as given for example by the statistics of natural amino acids or of natural amino acid sequences. In one aspect, during performing of the method of this disclosure, these pre-defined probability distributions may be adjusted, e.g., based on properties, such as the scores, of the sequence pool of the amino acid sequences, or based on recent scientific findings.

In contrast to previous approaches using trRosetta, the AF program is used to provide the structure prediction [4]. An optimization goal, i.e., the fitness function, is defined in terms of a mathematical function of the protein sequence, all-atom structure, and AF confidence. The fitness function may be composed from fitness function components, i.e., mathematical expressions. The composition of the fitness function from the fitness function components enables flexibly defining the fitness function according to structural properties and/or biological and/or physicochemical functions of the protein being designed. The optimization goal represents aspects of the design of the protein that is sought. The properties and/or the biological and/or physicochemical functions may relate to one or more of the structure of the protein being designed, binding properties of the protein being designed, a switching between conformations of the protein being designed, a structural alteration dependent on environmental conditions of the protein being designed, an amino acid distribution of the protein being designed (e.g., a human-like amino acid distribution for immune evasion), an amino acid sequence distribution of the protein being designed (e.g., a human-like amino acid sequence distribution), a function of the protein (e.g., enzymatic activity) including a predicted function predicted by a protein function predictor, a secondary structure of the protein being designed, an oligomeric state of the protein being designed, a propensity for post-translational modification (PTM) of the protein being designed including a predicted PTM predicted by a PTM predictor.

The first step in the prediction of the protein structure using AlphaFold is to search a large database of protein sequences (amino acid sequences) for similar protein sequences (similar amino acid sequences) of one or more input protein sequences (input amino acid sequences). The protein sequences may be of known origin and/or metagenomic origin. Once, similar ones of the input protein sequences are found, the newly found protein sequences are aligned against the input protein sequence. This alignment takes place by trying to match as many of the amino acids in the similar protein sequence as possible to corresponding ones of the amino acids in the input protein sequence. Unmatched amino acid sequences in the input protein sequence are assigned a gap “-”. This produces set of multiple sequence alignments. It will be appreciated that, at this stage, the aligned similar protein sequences do not have structure information associated with them. In a second, optional step, it is possible to input the structures of those aligned protein sequences for which the structure information is known into AlphaFold as structural templates.

The AF program processes, as the input protein sequence of the amino acids (i.e., input amino acid sequence), a single protein sequence (i.e., a single amino acid sequence) from the initial sequence pool (or initial pool of the amino acid sequences), together with a corresponding one of the multiple sequence alignment (as explained above), and the structure for the single protein sequence (or the single amino acid sequence) is predicted by constructing, using the multiple sequence alignment, an alignment of amino acids contained in that protein sequence alone [7]. The multiple sequence alignment predicts which of the amino acids contained in that protein sequence are located close to one another (see the discussion of predicted aligned error or pAE below). The method furthermore returns an all-atom structure for the single protein sequence (or the single amino acid sequence) and predicted confidence measures (see FIG. 1A, AlphaFold outputs). The value of the predicted confidence measure is expressed as a combination of the predicted local distance difference test (pLDDT) [39] and the predicted aligned error (pAE) [4]. The pLDDT test measures local model quality, while the pAE provides a measure of confidence for each amino acid pair in the protein sequence. The protein sequences are then optimized to maximize the fitness function (L) of the outputs (i.e., protein sequence, predicted all-atom structure and the afore-mentioned confidence measures predicted Local Distance Difference Test (pLDDT) and predicted Aligned Error (pAE)). This optimization can include minimizing the root mean square deviation (RMSD) to a reference structure for the protein and maximizing the AF prediction confidence. The RMSD is computed from the difference between the predicted structure of the protein from a known specific backbone structure of a protein. As the method is suitable for complex structure prediction [6], the fitness functions can also constrain oligomeric state cr conformational change upon protein binding (see FIG. 1 B). In general, any function of the protein structure, the sequence of amino acids and the prediction confidence may be used for the optimization.

For the protein sequence of the amino acids in the sequence pool, the value of the fitness function for the protein sequence of the amino acids is calculated. The protein sequences are further recombined and mutated by an optimizer to explore the sequence space of the proteins. The “mutation” means replacing one or more amino acids within the protein sequences by a different amino acid sampled uniformly at random. The “recombination” means choosing two sequences from the sequence pool and exchanging random stretches of both sequences at random. The sequence pool is updated with the recombined and/or mutated sequences.

An evolutionary algorithm is used to update the sequence pool, e.g., by mutating and/or recombining the protein sequences (amino acid sequences). Following [40] was used. However, other evolutionary algorithms or other gradient-free or gradient-based optimizers can be substituted as desired.

The evolutionary algorithm is conducted as followed. Using the random sequence pool of N sequences, the fitness function for the sequences of the amino acids was evaluated, yielding a fitness function values t associated with the sequences of the amino acids. In a next step, the amino acid sequence (or protein sequence) with the largest fitness function value tₘₐₓ was identified. In a subsequent step, the protein sequences in the sequence pool with a fitness function value t≥tₘₐₓ−tolerance·tₘₐₓ, for some tolerated suboptimality range (tolerance·tₘₐₓ) were selected until a certain number “M” of new amino acid sequences (or protein sequences) was identified. The selected amino acid sequences (or protein sequences) were further recombined, and mutated, and a so-called greedy improvement was performed on each recombined/mutated one of the amino acid sequences (or protein sequence). The term “greedy improvement” means that only those amino acid sequences (or protein sequences) which improve their fitness function value t over their non-mutated starting amino acid sequence (or protein sequence) were selected. Finally, the top N generated amino acid sequences (or protein sequences) were selected, whereupon the algorithm is repeated.

Throughout the optimization, the protein changes its structure and both local confidence measures, e.g., based on the pLDDT, and global confidence measures, e.g., based on the pAE, increase (as seen in FIG. 1 C). For a given protein design task, a threshold is selected above which the sequence of the amino acids for the protein (or amino acid sequence) is considered optimized. The amino acid sequences (or protein sequences) above this threshold are returned from the method and a subset of the returned amino acid sequences (or protein sequences) are submitted for validation using, e.g., molecular dynamics simulations and Rosetta ab initio structure prediction [21]. Only a subset is submitted to reduce the time required for processing the subset in the Rosetta program.

### Fitness Functions

The use of gradient-free methods enables the use of any function of the protein sequence of the amino acids (or amino acid sequence), all-atom structure, and AF confidence as the fitness function. This flexibility opens many possibilities for designing the amino acid sequences (or protein sequences and protein complexes with specific ones of the properties and/or the biological and/or physicochemical function. The fitness function may be specified to represent a goal of the design task.

The protein complex is a (possibly transiently) bound state of multiple ones of the proteins. In terms of this method, it reduces to simultaneously designing not a single protein sequence, but multiple ones of the protein sequences.

The method described in this document focusses on fitness functions making use of the afore-mentioned confidence models (or metrics) and protein backbone geometry (or backbone structure). This is, however, not limiting of the invention and other fitness functions can be used.

The confidence models return two main measures of confidence (or confidence metrics) for a protein structure prediction. As noted above, these measures of confidence are the predicted local distance difference test (pLDDT) [39] and the predicted aligned error (pAE) [4]. The pLDDT provides a measure of local confidence for each of the amino acids in the protein sequence. The pAE provides a predicted error for the position of the amino acids in the local coordinate frame of the other amino acids. For both confidence measures, the method predicts a binned distribution of values over the amino acids in the protein sequence. The pAE values are converted to predicted aligned confidence (pAC), which translates to pAC=1−μ_{pAE}/pAEₘₐₓ, where μ_{pAE} is the mean pAE and pAEₘₐₓ is the center of the highest pAE bin. Alternatively, it would be possible to work with the predicted template matching score (pTM) [41]. Optimizing the pLDDT and the pAE (e.g., high pLDDT and low pAE) provides a prior distribution over amino acid sequences, based on which native-like protein structures may be designed. A native-like amino acid sequence is an amino acid sequence, the sequence of amino acids (i.e., or string of amino acids) of which is larger than or equal to a minimum probability under a probability distribution observed in nature. Such probability distribution observed in nature may be determined based on sequences and structures stored in PDB. A native-like amino acid sequence thus has a

The fitness functions may include a term maximizing the value in the confidence level or confidence metric given by:

$${L}_{conf} ( X ) = \frac{{μ}_{pAE} + {μ}_{pLDDT}}{2}$$

where X=(x, pAE, pLDDT) is a tuple containing the all-atom structure x, as well as the values of pAE and pLDDT for the protein sequence, μ_{pLDDT} is the mean of the pLDDT, and μ_{pAE} is the mean of the pAE.

The AF returns protein all-atom coordinates [4], which enables in a further aspect of the method to introduce geometrical (or structural) features into the fitness function. It is possible to introduce the fitness function components, i.e., mathematical expressions, into the fitness function which depend on arbitrary geometrical features of the protein sequence and its predicted structure. These geometrical features are any geometrical features of the protein being designed that need to be optimized. For example, the geometrical features include, but are not limited to:
any weighted sum/product of AlphaFold confidence measures
protein secondary structure as determined from the predicted 3D structure
protein radius of gyration
difference of the protein structure to some reference protein structure
distribution of amino acids within the protein sequence
protein solubility as predicted from the protein sequence and structure
protein function, e.g., physicochemical and/or biological function, as predicted by another model from the protein sequence and structure
for protein complexes: relative positions of the centers of mass of each of the constituent monomers
or alternatively, any weighted sum/product of these geometrical features.

The main building block for the fitness function components to be introduced into the fitness function, are measures of difference between two sets of coordinates. The following protein structure metrics to be used within the fitness function are:

Aligned error [4] is given by:

$$A {E}_{ij} ( x , y ) = ‖ {T}_{xi} {x}_{i} - {T}_{yj} {y}_{i} ‖$$

where T_{x, I} is the coordinate transformation moving a vector to the local coordinate system at backbone atom xᵢ. This is the aligned error used in AF training [4].

The Frame-Aligned Point Error (FAPE) is given by [4]:

$$FAPE ( x , y ) = \frac{1}{c {N}^{2}} {\sum }_{i j}^{N} clip ( {AE}_{l j} , c )$$

where c is a cutoff for the maximum error, N is the number of amino acids in the protein sequence and clip is a function which cuts off values at a maximum cut-off. In other words, clip (AE, c) sets all values in the aligned error which greater than the value c to the value c. The Frame-Aligned Point Error is the main loss function used for training AF. The FAPE is a function which computes the difference between two protein structures, and is invariant to global rotation and translation, but not to reflection. The smaller a FAPE value between two protein structures, the more similar those structures are. FAPE is zero if and only if the protein structures being compared are equal up to global rotation and translation. The training of AF uses a true structure determined, for example, from X-ray crystallography.

The distance RMSD is given by

dRMSD(x,y)=RMSD(d(x),d(y))

The function d(x) computes the distances between each pair of alpha carbons in the backbone of the amino acid and is calculated as follows. Given a protein structure, take the 3D coordinates “x” of its backbone alpha carbon atoms (i.e., the atom in the amino acid to which the side chain is attached). The function d(x) computes the distances between each pair of alpha carbons. In practical terms, this function provides enough information to recover the 3D coordinates of all of the alpha carbon atoms up to global translation, rotation, and reflection, which is why pairwise distances may represent the actual 3D coordinates in the fitness function. If the pairwise distances for two structures are the same, the actual 3D coordinates will be the same up to global translation, rotation, and reflection of the protein sequence.

The Template Matching score [41] is the measure of structural similarity between the protein structures:

$${L}_{T M} ( x , y ) = {max}_{j} \frac{1}{N} {\sum }_{i}^{N} f ( {AE}_{i j} ( x , y ) )$$

$$where$$

$$f ( d ) = \frac{1}{1 + {( \frac{d}{{d}_{0} ( N )} )}^{2}}$$

$$and$$

$${d}_{0} ( N ) = 1.24 \sqrt[3]{max ( N , 19 ) - 15} - 1.8$$

L_{TM}(x,y) is an approximation of the template matching score which does not require structural alignment [4]. The measure of the template matching score is normalized to one and provides a smooth fitness function for highly dissimilar structures.

Furthermore, it is possible to enforce general shape constraints for proteins using a compactness fitness function L_{comp}, which penalizes large protein radii:

$${L}_{comp} ( X ) = \frac{- 1}{N} {\sum }_{i}^{N} \vert {x}_{i} - {μ}_{x} \vert - {max}_{i} \vert {x}_{i} - {μ}_{x} \vert$$

where μₓ denotes the center of mass of the coordinates.

Monomers. To generate globular monomeric proteins, the confidence fitness function (or confidence metric) was combined with a weighted one of the compactness fitness function as follows:

$${L}_{mon} ( X ) = 2 {L}_{conf} ( X ) - \frac{1}{15 N} {L}_{comp} ( X )$$

This trades off compactness for confidence in the case of elongated structures. It will be appreciated, however, that this method can be used for other protein designs and is not limited to globular monomeric proteins. The fitness function which evaluates the globularity of a protein is relatively straight-forward. This is not as straight-forward to do for deciding if a part of the protein is, for example, a trans membrane domain. Given a fitness function which decides this from protein sequence and/or structure, the method could be used to design other types of proteins.

Protein Complexes. For the design of the protein complex design, the same fitness function Lₘₒₙ was used for the constituent monomers in the protein complex. This ensures that each monomer has a stable high-confidence structure. Previous work identified inter-monomer pAE as a predictor of complex formation [19]. A low inter-monomer pAE (or equivalently, a high inter-monomer pAC) corresponds to a high confidence in complex prediction. As noted above, pAE is the predicted error in distance and relative orientation for each pair of the amino acids in the protein sequence. Assuming prediction is calibrated (i.e., predicted uncertainty corresponds well to true uncertainty), a low predicted error pAE in inter-monomer residue pairs corresponds to a highly constrained distance and relative orientation between the monomers. This is the case only when the proteins in the protein complex are interacting with each other. For non-interacting ones of the proteins, uncertainty in distance/relative orientation is high as the proteins are in no way constrained in their distance/orientation relative to each other.

Therefore, another combination of the confidence fitness function and the compactness fitness function is imposed on the complex protein, resulting, e.g., in:

$${L}_{cpx} ( X , {X}^{m} ) = \frac{1}{M + 1} ( 2 {L}_{conf} ( X ) + {L}_{comp} ( X ) + {\sum }_{i}^{M} {L}_{mon} ( {X}^{m} ) )$$

where X denotes the predictions for the all-atom structure of the complex protein and X^{m} denotes the all-atom structure for each monomer m.

Conformational change. To steer towards conformational change upon complex formation of the protein complexes, a conformational change fitness function L_{CC} is defined. This fitness function L_{CC} may be added to the fitness function L_{cpx}(X, X^{m}) defined above:

$${L}_{cc} ( X , {X}^{m} ) = \frac{1}{M} {\sum }_{m}^{M} ( 1 - TM ( monomer ( x , m ) , {x}^{m} ) )$$

where monomer (x, m) extracts the coordinates of the monomer m from the structure of the oligomer x., maximizing this fitness function minimizes the TM score [41] between the protein structure as part of the protein complex and its structure as a monomer.

Protein-protein interactions. Interface pAE (ipAE) correlates with the probability of protein-protein interaction. Optimizing for low ipAE between two protein monomers allows to design protein-protein interactions. In combination with additional confidence and geometric fitness function components which impose constraints (see TABLE 1 below), it is possible to define the fitness function for de novo monomer, oligomer (L_{denovo}) as well as protein binder design (L_{binder}). It is possible to search for sequences for which AF predicts different monomeric and oligomeric states by designing a corresponding fitness function (L_{change}).

The fitness function components of the fitness function include multiple confidence measures (e.g., pAE, interface pAE, pLDDT). The fitness function components of the fitness function include geometric constraints on the predicted protein structure (radius of gyration R_{g}, maximum distance d(Xᵢ,Xⱼ) between pairs of residues Xᵢ and Xⱼ). Confidence fitness function components provide a prior distribution over amino acid sequences that ensures generated structure candidates are native-like (L_{conf}) and exhibit desired protein-protein interactions (L_{ipAE}). The geometric constraints constrain the predicted protein structure to conform to a designed topology and/or geometry.

In addition to the afore-mentioned components for the fitness function, further ones of the components are listed and described in the table below.

TABLE I

| Fitness function | | |
|---|---|---|
| component | Mathematical expression | Description |
| L_{pAE}(X) | $1 - \frac{mean ( pAE ( X ) )}{p {AE}_{max}}$ | mean normalized predicted alignment error |
| L_{ipAE}(X, Y) | $1 - \frac{mean ( ipAE ( X , Y ) )}{p A {E}_{max}}$ | mean normalized interface confidence over X and Y |
| L_{pLDDT}(X) | mean(pLDDT(X)) | mean pLDDT over X |
| Lbind (X\|T; α) | α · L_{ipAE} (X, T) + (1 − α) · L_{conf} (X) | interaction confidence |
| | | fitness function for a binder |
| | | X against a target protein T |
| Lₛᵢₜₑ (X\|T; r; s) | max (r, minᵢ (d(Xᵢ, Xⱼ))) − r | thresholded minimum |
| | | distance of amino acids in |
| | | binder X to the target site |
| | | T_{S} on the target protein T |
| L_{compact}(X) | meanᵢ (d (Xᵢ, meanⱼ(Xⱼ))) + | thresholded constraint on |
| | maxᵢ (d (Xᵢ, meanⱼ(Xⱼ))) | the radius of gyration |
| L_{R}(X; Rg) | $max ( {R}_{g} , \frac{1}{{N}^{2}} {\sum }_{ij} d ( {X}_{i} , {X}_{j} ) ) - {R}_{g}$ | thresholded constraint on the radius of gyration |
| L_{denovo}(X¹, . . . , X^{N}) | $\frac{1}{N + 1} ( {L}_{conf} ( {X}^{i} : … : {X}^{N} ) + {\sum }_{i} {L}_{conf} ( {X}^{i} ) )$ | de novo design fitness function |
| L_{change} (X,X:Y; α) | $α \cdot ( 1 - {L}_{TM} ( X , {X}_{X : Y} ) ) + ( 1 - α ) \frac{{L}_{conf} ( X ) + {L}_{conf} ( X : Y )}{2}$ | conformational change fitness function used in this work. X_{X:Y} denotes the |
| | | structure of X in the com- |
| | | plex X:Y |
| L_{binder} (X\|T; s) | ${L}_{bind} ( X \vert T ; 0.7 ) - {L}_{site} ( X \vert T ; 6 Å ; s ) - 0.1 \cdot {L}_{R} ( X )$ | |

Input representation. The amino acid sequences (or protein sequences of the amino acids) are represented as one-hot encoded arrays with 20 classes, one class for each standard amino acid. This allows the use of both gradient-free and gradient-based optimizers, as the gradient can be estimated through a one-hot representation using a straight-through estimator or similar [42]. As input to AF, an additional multiple-sequence alignment is constructed with only the single input sequence, as well as blank template features. The blank template features are empty template features. Instead of passing one or more templates as input to AlphaFold, no templates are passed at all to AlphaFold. However, AlphaFold still expects an input which contains information about the templates. The use of the term “blank” template features means the template input which actually contains no templates.

Sequence templates. To reduce search space size and implement exact sequence constraints without introducing additional fitness functions, the optimized amino acid sequences (or protein sequences of the amino acids) are represented as a pair (S, T) where S: [symbol] is a one-hot representation of all V variable amino-acids and T: [symbol] → [symbol] is a template mapping which assembles the Vvariable amino acids into the final evaluated amino acid sequence (or protein sequence) of length N. For example, to design a homodimer with monomer size 64, the actual number of variable amino acids is V=64 and the template T simply concatenates the input sequence S: [symbol] with itself.

Complex representation. AF was originally designed to accept only single protein sequences as an input. In order to enable AF to predict the structure of the protein complexes, the principles set out in [7] were followed. In this case, chain breaks for prediction of the structure of the complex protein are represented by introducing an increment greater than 32 to the residue indices of each additional chain beyond the first chain. This increment is used to separate the residues on the different chains by more than 32, which is the maximum relative residue index difference separately embedded by AF [4]. In addition, following [7] the multiple-sequence alignment features are split, such that each monomer has a separate copy of its sequence alignment features, with gaps at the sequence positions of the other monomers.

This can be explained by analog using a sequence (in a pseudo-fasta format): Suppose the sequence is the following

>1
MERRYCHRISTMAS
>2
HAPPYNEWYEAR
>3
HAPPYEASTER

Let us suppose that AlphaFold should predict the structure of their complex, the input to AF would have the following multiple sequence alignment (in the pseudo-fasta format):

>1:2:3
MERRYCHRISTMASHAPPYNEWYEARHAPPYEASTER
>1
MERRYCHRISTMAS-----------------------
>2
--------------HAPPYNEWYEAR-----------
>3
--------------------------HAPPYEASTER

where “-” denotes a gap (i.e., an unmatched amino acid) in the multiple sequence alignment. That is, for a complex of N proteins, a multiple sequence alignment of N+1 sequences is provided. The first one of the sequences is the sequence concatenating all of the single protein sequences. The k+1th sequence is the sequence of the kth input protein, with a number of gaps (“-”) to the left equal to the total length of the 1st to k−1th protein and a number of gaps (“-”) to the right equal to the total length of the k+1th to Nth proteins.

### Design Studies

Optimization. For the optimization, an evolutionary-strategy optimizer following [40] was used, as described above. Population size was set as ten. During mutation, the population size was expanded by a factor of two. The protein sequences of the amino acids with a suboptimality of at most 10% (in other words, the value of the tolerance range, as described above was set at 0.1) were considered for mutation and recombination. The recombination was applied by crossover with probability of 10% at each position in the sequence of the amino acids. In this context, crossover means taking two amino acid sequences and creation of the recombined sequence is done by initially taking amino acids from the first sequence. At each amino acid, with a probability of 10% switch the sequence from the one at which the amino acids are currently being taken to the other sequence.

The optimization was considered complete for those sequences with L>1.5 for Lₘₒₙ, L_{cpx}, and L>0.9 for the fitness functions including L_{cc}. TABLE II and TABLE III contain further parameters of optimization runs performed.

AlphaFold configuration. For optimization, AF was configured for single sequence uses by disabling ensembling templates, extra MSA features and restricting the number of MSA features to the number of monomers modelled. The number of AF iterations (recycling steps) was kept as a parameter for each optimization run (see TABLE II and TABLE III below). For the larger protein complexes, the number of iterations was decreased to two to speed up the computation. The parameter set model_1_ptm was used for all of the experiments.

TABLE II
De novo protein design runs and parameters.

| | | | | # | |
|---|---|---|---|---|---|
| | Sequence | | Is a | AlphaFold | fitness |
| Name | length | #Monomers | homooligomer? | iterations | function |
| monomers 32 | 32 | 1 | — | 4 | Lₘₒₙ |
| monomers 64 | 64 | 1 | — | 4 | Lₘₒₙ |
| monomers 128 | 128 | 1 | — | 4 | Lₘₒₙ |
| monomers 128 | 128 | 1 | — | 4 | Lₘₒₙ |
| homodimers 32 | 32 | 2 | yes | 4 | L_{cpx} |
| homodimers 64 | 64 | 2 | yes | 4 | L_{cpx} |
| homodimers 128 | 128 | 2 | yes | 2 | L_{cpx} |
| heterodimers 32 | 32 | 2 | no | 4 | L_{cpx} |
| heterodimers 64 | 64 | 2 | no | 4 | L_{cpx} |
| heterodimers 128 | 128 | 2 | no | 2 | L_{cpx} |
| trimers 32 nr4 | 32 | 3 | yes | 4 | L_{cpx} |
| trimers 64 nr4 | 64 | 3 | yes | 4 | L_{cpx} |
| trimers 32 nr2 | 32 | 3 | yes | 2 | L_{cpx} |
| trimers 64 nr2 | 64 | 3 | yes | 2 | L_{cpx} |
| tetramers 32 nr4 | 32 | 4 | yes | 4 | L_{cpx} |
| tetramers 64 nr4 | 64 | 4 | yes | 4 | L_{cpx} |
| tetramers 32 nr2 | 32 | 4 | yes | 2 | L_{cpx} |
| tetramers 64 nr2 | 64 | 4 | yes | 2 | L_{cpx} |
| pentamers 32 nr4 | 32 | 5 | yes | 4 | L_{cpx} |
| pentamers 64 nr4 | 64 | 5 | yes | 4 | L_{cpx} |
| pentamers 32 nr2 | 32 | 5 | yes | 2 | L_{cpx} |
| pentamers 64 nr2 | 64 | 5 | yes | 2 | L_{cpx} |
| conformation | 64 | 2 | no | 2 | 0.7 · L_{cpx} + |
| change dimers 64 | | | | | 0.3 · L_{cc} |
| conformation | 32 | 3 | yes | 2 | 0.7 · L_{cpx} + |
| change trimers 32 | | | | | 0.3 · L_{cc} |

TABLE III
Binder design runs and parameters.

| | Binder | Target | # AlphaFold | fitness |
|---|---|---|---|---|
| Name | length | protein | iterations | function |
| binders | 64 | monomers 64 | 4 | L_{cpx} |
| 64-64-0 | | result 0 | | |
| binders | 128 | monomers 64 | 4 | L_{cpx} |
| 64-64-2 | | result 2 | | |

### Rosetta Validation

The designed proteins sequences were validated using the Rosetta suite of protein design and structure prediction tools [21]. The fragment-assembly-based ab initio structure prediction

was used as an independent baseline (i.e., independent method) for the designed protein sequences and the protein structures.

The ab initio structure prediction is carried out as follows. The protein secondary structures were predicted using PSIPRED [44] and S4PRED [45] to provide the protein secondary structures for fragment selection. 3-mer and 9-mer fragments were selected from the Rosetta fragment database based on the secondary structure and the protein sequence information. Alignment information was not used, as the designed protein sequences did not have sufficient homology to the natural protein sequences. The Ab initio structure prediction was performed by fragment-assembly using the Abinitio Relax protocol. Two starting conformations were evaluated. The first starting conformation started from an extended conformation in which all backbone torsion angles are set at 1800 (in practice this is the default starting conformation for the ab initio structure prediction using the Abinitio Relax protocol) and the second starting conformation started from the AF predicted structure.

It will be appreciated that there is no a priori information about the structure of the protein for ab initio structure prediction. The process of the prediction needs to start from some initial conformation, which is a theoretically valid conformation of an amino acid chain. This initial conformation does not necessarily have to be the extended conformation, but the extended conformation is convenient as an initialization as it is easy to set up. There are also no clashes in the protein backbone in this initial conformation, as opposed to, e.g., starting from a random conformation. It will be noted, however, that already within a few steps of the ab initio structure prediction process, the structure will have changed to something more like an actual protein structure.

For both runs of the two starting conformations, 32000 decoys (conformations having a low free energy) were generated. The decoys from both of the runs were pooled and scored using the Rosetta all-atom energy function [46]. The overall ten lowest energy decoys were selected for relaxation. The decoys were relaxed using the Relax protocol. The AF predicted structure was also relaxed to generate ten additional decoys in case the ab initio structure prediction failed to find a minimum energy structure. The decoys were rescored using the same Rosetta score function to compute directly comparable energies. It will be appreciated that ab initio structure prediction and relaxation use different score functions in Rosetta. Thus, in order to be able to compare the quality of structures between both of these protocols, the output protein structures need to be scored again (rescored) using the same score function. The lowest energy relaxed decoy was selected as the final predicted structure and its Rosetta score and RMSD with respect to the AF predicted structure were evaluated.

Protein-protein interface analysis. As Rosetta does not allow for the ab initio structure prediction of protein complexes other than in the case of symmetric homo-oligomers, interfaces of designed complexes were assessed using the Interface Analyzer protocol [47]. The protein structures were relaxed, and the Rosetta binding energy was computed by removing a single monomer from the complex, followed by repacking. As a further measure of interface quality, packing statistics were computed using the PackStat protocol [48], which detects solvent-inaccessible unfilled regions within an interface.

Molecular dynamics simulation-based validation

A subset of the designed monomers of the protein sequences and the higher-order protein complexes were further validated by performing all-atom molecular dynamics (MD) simulations in explicit solvent and analyzing the corresponding properties of structural flexibility and internal/intramonomeric and interfacial contacts (for multimeric proteins and protein complexes). The simulation carried out in explicit solvent means that the proteins (or the protein complexes) are “put into a box” in which every molecule of water and every salt ion is included explicitly. This term contrasts with “implicit solvent” methods, in which the water molecules and the ions are not simulated explicitly but are instead modelled by modifying the force field.

Initial system construction. The standard AMBER force field (ff14sb) was used to describe all protein parameters [49]. Each protein or protein complex was solvated using atomistic TIP3P water with a minimum of 10 Å of padding to form a cubic periodic box and then electrically neutralized with an ionic concentration of 0.15 M NaCl that used standard ionic parameters.

Simulation protocol A standardized minimization, equilibration and simulation protocol comprising eleven stages was developed for the systems of solvated protein structure and the solvated protein complex structures. A set of restraints (RS) were applied to each system at specified stages of equilibration. These restraints comprise restraining all heavy (i.e., non-hydrogen) atoms of the proteins. Each system was subsequently minimized across four stages with 1500 steps (500 steepest-descent+1000 conjugate-gradient) of minimization applying restraints RS with different force constants in each sequential stage: Stage 1: 10 kcal/molA², Stage 2: 5 kcal/molA², Stage 3:1 kcal/molA², Stage 4: unrestrained. The MD simulations were performed in all subsequent stages. The SHAKE algorithm was employed on all atoms covalently bonded to a hydrogen atom. A time-step of 2 fs was used. The long-range Coulomb interaction was handled using a GPU implementation of the particle mesh Ewald summation method (PME) [50]. A nonbonded cut-off distance of 10 Å was used. In Stage 5, each system was heated from 10 K to 300 K in 1 ns and with RS (k=10 kcal/molA²). The temperature was subsequently maintained at 300 K using a Langevin thermostat with a damping constant of γ=5.0 ps⁻¹ and in Stage 6 the systems equilibrated for 1 ns at constant volume, thus in the NVT ensemble (i.e., constant number of particles N, constant volume V and constant temperature T). Subsequently the pressure was maintained at 1 atm using a Berendsen barostat with a pressure relaxation time of τₚ=1.0 ps and the systems simulated in the NPT ensemble for 100 ps for each of the subsequent stages with RS: Stage 7: k=10 kcal/molA², Stage 8: k=5 kcal/molA², Stage 9: k=1 kcal/molA², Stage 10: k=0.5 kcal/molA². Finally in Stage 11, the RS restraints were removed, and the systems simulated in the NPT ensemble for a further 5 ns. Following this, a production simulation of 100 ns each was performed for each system in the NPT ensemble with the same conditions as Stage 11. Coordinate snapshots from production simulations were generated every 10 ps, resulting in a trajectory of 10,000 snapshots per system.

Structural flexibility and contact analysis. Structural stability and flexibility were analyzed by computing the root-mean squared deviation (RMSD) of the backbone atoms of the protein with respect to the initial predicted protein structure (after aligning the protein backbone) as well as the root-mean-squared fluctuation (RMSF) with respect to the average protein structure in the production MD. In the case of the protein complexes, this analysis was conducted both on the overall complex protein and for the individual monomer proteins separately. The number of sidechain-sidechain intramonomer contacts were computed for each snapshot of the production MD, based on a heavy atom distance threshold of 4 Å. Similarly, the interfacial contacts were computed for each interface in the simulated protein complexes based on the same criteria. An overall picture of the protein contacts was provided by summing the total intramonomer and interfacial contacts.

The intramonomer contacts are the contacts within the monomer. These intramonomer contacts provide an indicator of stability of the monomer. If, throughout the MD simulation the number of the intramonomer contacts decreases, this indicates an instability of the protein, i.e., the protein is pulling apart. The interfacial contacts represent the intermonomer contacts between each pair of the monomers. These interfacial contacts indicate stability of the protein-protein interaction. If, throughout the MD simulation the number of the intermonomer contacts decreases, this indicates that the monomers dissociate from each other.

Finally, for all simulated systems, an approximate potential of mean force (G) was determined by computing the Boltzmann-weighted distribution (G=−k_{B}T ln(ρ) in the 2D collective variable space of the global RMSF and total contacts (total intramonomer+total interfacial) contacts from the last 50 ns of simulation, where p is the normalized frequency in the binned 2D landscape.

### Structural Clustering

The designed protein sequences of length 64 amino acids and larger were subdivided into a dictionary of fragments using Geometricus [51]. The fragments were collected using the k-mer method with k=16 and the radius method with cut-off of 10 Å. The fragments are shorter chains, or local neighborhoods of the amino acids. The Fragments collected using the radius method contain all amino acids around a given amino acid where at least one atom is within the cut-off radius. Realistically, this will be the amino acids in the chain around the given amino acid, as well as other amino acids which are close in the 3D structure, but not necessarily in the amino acid sequence.

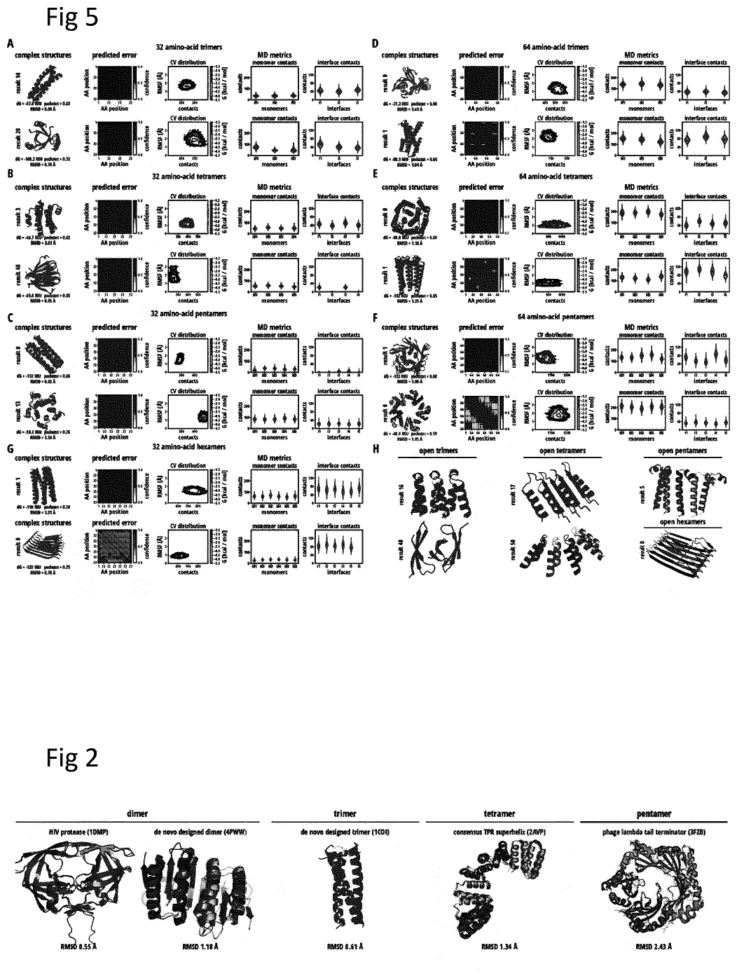

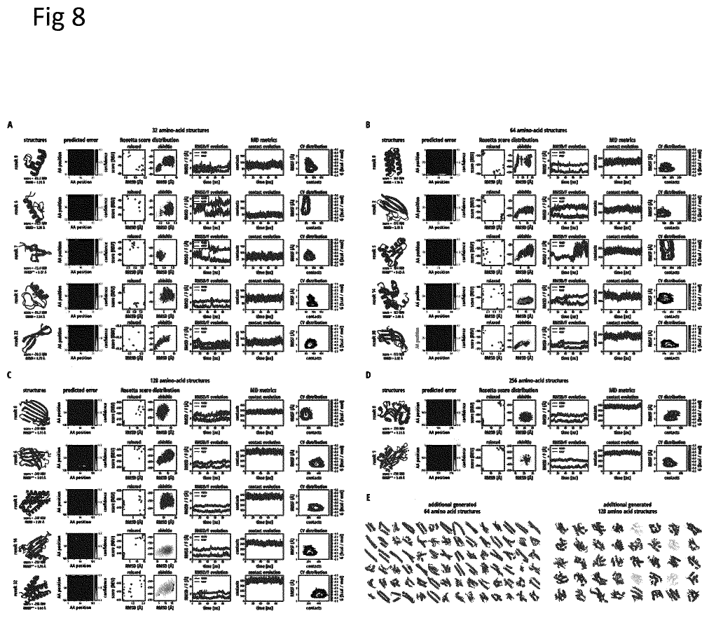

A collection of features representation was computed for all designed proteins and dimensionality was reduced using non-negativematrix factorization (NMF) with 50 components [52]. This computation is carried out as follows. The number of fragments in each protein structure corresponding to each fragment cluster identified by Geometricus was counted. A vector of these counts is returned, and this vector is the bag of features representation. The proteins were separated into ten clusters using ward-linkage agglomerative clustering on their NMF components. Results. It was found that AlphaFold predicts the structure of protein complexes as recently shown by [7], AF as trained on the single-chain protein structures can be used for protein complex structure prediction. To gain confidence in the suitability of AF to design the protein complexes using the method of this document, the structure of small protein dimers, trimers, tetramers and pentamers was first predicted (FIGS. 2 and 8 The AF predictions show low root-mean-squared deviation (RMSD) to the native structure<3 Å for the protein complexes considered. While this may be due to these structures being part of the PDB and thus part of AF's training dataset[4], together with recent work on protein complex prediction with AF [6], it provides some indication that AF could be suitable for the design of protein complexes using the method of this document.

### Single Sequence Optimization Designs Diverse Monomers

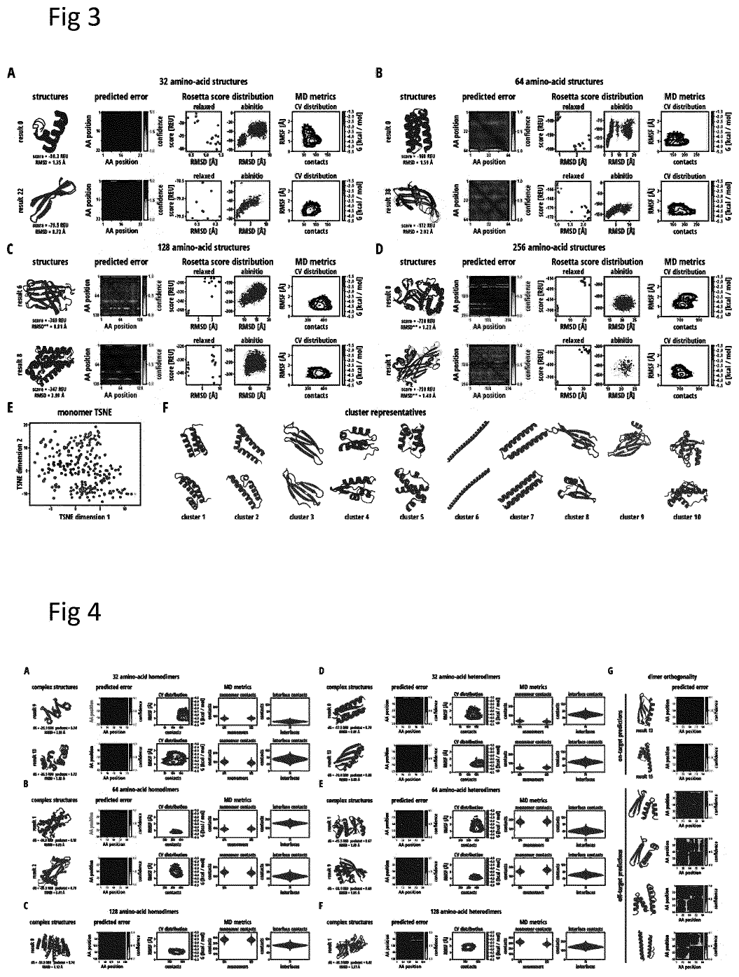

As an initial step towards AF-based de novo protein design, the amino acid sequences of length 32, 64, 128 and 256 amino acids to design monomeric, globular proteins (FIG. 3) were carried out. The designed sequences of the amino acids for the monomers show a high predicted aligned confidence (FIG. 3, predicted error), exceeding a mean predicted aligned confidence of 0.82 and mean pLDDT of 0.83 (normalized to the range [0, 1]). This indicates that AF assigns high confidence to the predicted structure, as pLDDT of 0.7 or higher corresponds to a confident prediction of backbone structure [4, 5]. To validate the AF-predicted structure, fragment-based ab initio structure prediction using Rosetta [21] was performed for a randomly chosen subset of the designs, starting from an extended amino acid chain, as well as from the AF structure (see FIG. 8). For designed protein sequence of each of the monomers, the ten lowest-energy Rosetta structures was extracted. These extracted structures of the monomers as well as the AF structure using the Rosetta force field [46] was extracted and the lowest-energy structure was chosen. For most of the structures of the monomers, the RMSD between the AF structure (grey, FIG. 3 (A-D), structures) and the lowest-energy structure (red) is less than 3.0 Å. For those protein structures with distances exceeding this threshold (FIG. 3, C: result 8) the larger deviation seems to be due to a flexible α-helix and the AF predicted structure has a comparably low Rosetta energy.

To visualize the energy landscape of folds for each of the designed monomer, the distribution of decoys was inspected from ab initio folding with their Rosetta energy [46] and RMSD to the AF structure (FIG. 3 (A-D), Rosetta score distribution). It was noted that for monomers of size 32 and 64 amino acids, relaxations of the predicted structure (blue) have comparable, yet higher energies compared to the best 10 decoys from ab initio prediction (red) (FIG. 3 (A, B), Rosetta score distribution, relaxed). Strikingly, for monomers of size 128 and 256 amino acids, the relaxed AF structures (blue) exhibit far lower energies compared to the ab initio decoys (red) (FIG. 3 (C, D)). This indicates a failure of the ab initio structure prediction to find a reasonable minimum energy structure for those monomers. Indeed, the distribution of decoys for the monomers of size 32 and 64 amino acids shows funnel shaped distributions with a single minimum at both low energy and low RMSD characteristic for protein folding (FIG. 3 (A, B), Rosetta score distribution, ab initio). In contrast, for sizes 128 and 256 amino acids, the distribution is much more diffuse with no clear minimum being visible, indicating failure of ab initio prediction to find a reasonable minimum (FIG. 3 (A, B), Rosetta score distribution, ab initio). However, the observation that relaxed AF structures exhibit very low Rosetta energy [46], increases confidence that designed structures are plausible.

As a further validation step, the structural stability of the designed monomers was assessed by performing all-atom molecular dynamics (MD) simulations. The Boltzmann-weighted frequency distribution in the 2D collective variable (CV) landscape consisting of backbone RMSF and intramonomer contacts shows that most systems exhibit a well-defined unimodal distribution centered on low RMSF and a significant number of contacts. This suggests the vast majority of conformers within the production ensemble sample a narrow range consistent with a maintenance of structural stability (FIG. 3 (A-D), MD metrics). Furthermore, the time evolution of the backbone RMSD with respect to the initial AF-predicted structure and backbone RMSF with respect to the MD-averaged structure are stably below 4 Å and 2 Å respectively in the majority of monomer systems (see FIG. 8). Similarly, the time evolution of the intra-monomer contacts remains stable for the simulated monomers, suggesting a maintenance of folded structure. As expected, the number of contacts grows with monomer size ranging from ˜50-100, ˜100-200, ˜300-400 and ˜600-800 for monomer lengths 32, 64, 128 and 256 amino acids respectively (see FIG. 8).

To assess the diversity of structures generated by the method of this document, the monomers of size 64 amino acids or larger were clustered and representative structures for each cluster extracted (FIG. 3 (E, F)). The structures were embedded using Geometricus [51], agglomerative clustering performed, and their distribution visualized (FIG. 3I)). By visual inspection, the cluster representatives encompass α-only (clusters 1, 2, 5, 6, 7), β-only (cluster 3) and mixed αβ proteins (clusters 4, 8, 9, 10) (FIG. 3 (F)).

### Searching for Sequence Pairs Enables De Novo Dimer Design

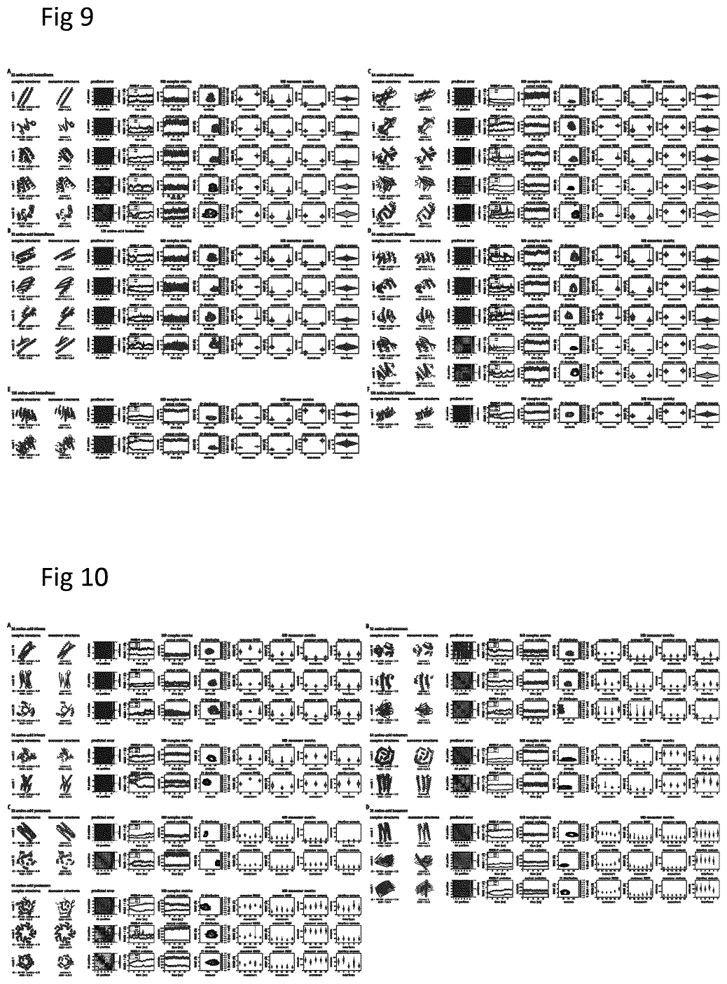

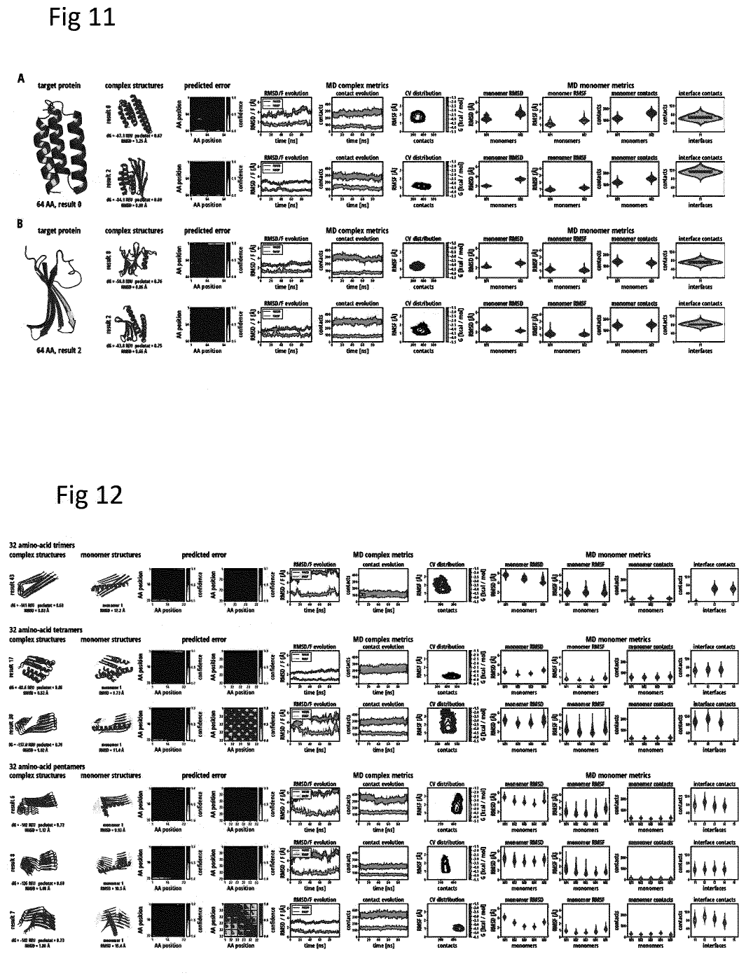

The method was next applied to de novo design of protein homodimers and heterodimers. The dimers with monomer size 32, 64 and 128 amino acids were optimized. As in the previous monomer designs, the predicted dimers show high predicted aligned confidence for the AF structure (FIG. 4 (A-D), predicted error) with mean pAC>0.75 and mean pLDDT>0.83. By visual inspection, the designed interfaces show a high level of shape complementarity. In lieu of the ab initio structure prediction, which is in general harder to access for protein complexes using Rosetta, the Rosetta binding energy was computed between the monomers, as well as a score of interface packing statistics [48] as measures for structure quality. This validation was applied to a subset of designed dimers (see FIG. 9). The dimers were relaxed using the Rosetta forcefield [46] noting that all relaxed structures have a low RMSD with respect to the AF-predicted structure (FIG. 4, (A-F), complex structures). Furthermore, most of the dimers exhibit a packing statistic score greater than 0.6, which is comparable with the packing statistic of crystal structures with resolution 2.0 Å [90, 103]. Most structures show a Rosetta binding energy better than −40 REU hinting that predicted interfaces are stable under Rosetta [46].

The MD simulations of dimer and higher order oligomer systems allow analysis of both intramonomer contacts as well as interfacial contacts between monomers. The Boltzmann-weighted frequency distributions in the CV space of global RMSF and total contacts (intramonomer and interfacial) mostly show unimodal distributions with low mean RMSFs (<2 Å) and significant numbers of total contacts that increase with complex size (FIG. 4, (A-F), MD metrics). Many of the dimer systems show stable time evolution of global RMSDs and RMSFs (see FIG. 9), although significant deviation is observed in a few of the dimer systems. Dissection of RMSDs, RMSFs, intramonomer and interfacial contacts, shows that both the monomers in either the homodimer or heterodimer systems exhibit similar flexibility as well as number of intramonomer contacts. There are significant numbers of interfacial contacts in each dimer system, although, as expected these are fewer than the corresponding intramonomer contacts.

For synthetic biology applications, orthogonality is a desirable property for dimers [16]. That is, pairs of monomers designed to dimerize should only bind their designed partner, not monomers of other designed dimers. As a preliminary test for orthogonality of dimers designed using the method, all combinations of monomers for a pair of designed dimers of monomer size 32 amino acids were predicted (FIG. 4 (G)). On-target complexes were predicted by AF with high confidence (FIG. 4, (G), on-target predictions). In contrast, for off-target complexes—that is, complexes of monomers not designed for dimer formation—the mean predicted aligned confidence for inter-monomer amino acid pairs dropped below 0.5. This indicates that AF cannot confidently predict these off-target combinations as a dimer, providing preliminary evidence that dimers designed using the method outlined in this document can indeed exhibit orthogonality.

### Multiple-Sequence Optimization Allows for Homo-Oligomer Design

To demonstrate the feasibility of protein complex design beyond dimers, a design of homo-oligomers from trimers to hexamers was carried out. Monomer sizes of 32 and 64 amino acids were considered for trimers, tetramers, pentamers and 32 amino acids for hexamers. As before, AF predicted aligned confidence was evaluated, Rosetta binding energy, packing statistics and behavior under 100 ns of molecular dynamics simulations for a subset of oligomers (see FIG. 10). Designed structures show high predicted aligned confidence with most structures at pAC and pLDDT>0.7 (FIG. 5)

(A-G) predicted error), low RMSD to the Rosetta relaxed structure<2.0 Å, good binding energy<−40 REU and packing statistics>0.59 comparable to natural protein complexes [53] (FIG. 5 (A-G) complex structures).

MD simulations of homo-oligomers exhibit similar properties to the dimers, with time evolution showing generally modest RMSDs (≤6 Å) and RMSFs (≤2 Å) and significant numbers of intramonomer and interfacial contacts (see FIG. 10). Boltzmann-weighted distributions in the RMSF-contact space again show unimodal distributions with mean RMSFs remaining around 2 Å and total contacts scaling with complex size. Dissection of individual intramonomer and interfacial contacts shows notable contacts in each monomer and interface for almost all systems (FIG. 5 (A-G) MD metrics).

Interestingly, inspection of the designed protein complexes reveals a subset of extended oligomers with exposed interfaces on both sides (FIG. 5 (G-H)). The designed sequences of the amino acids could potentially oligomerize into larger assemblies. Correspondingly, the MD simulations of these complexes show maintenance of intramonomer contacts for all monomers and sequential interfacial contacts for all but one interface, as expected. This indicates the possibility of designing and validating large-scale assemblies in AF beyond simple oligomers.

Designing with a Fixed Monomer Finds Binders for Target Proteins

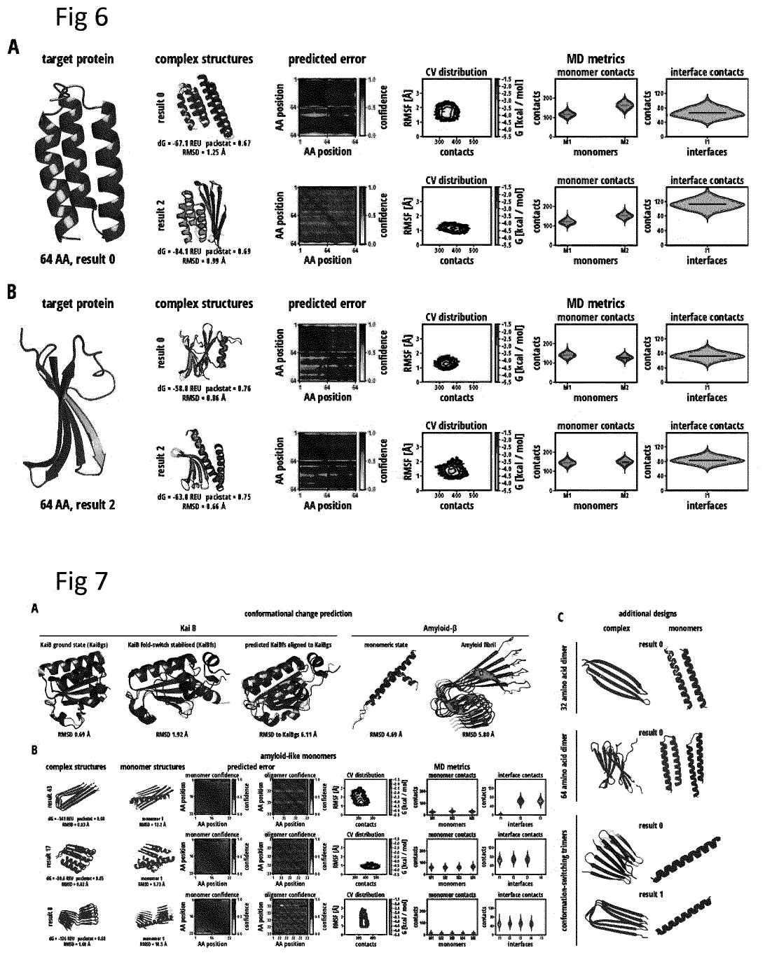

As a natural extension to the dimer and oligomer design using AF with potential applications in biologics design and synthetic biology, proteins binding to a fixed target protein (e.g., a fixed monomer, also referred to as template) were designed (FIGS. 6 and 12, Two proteins of length 64 amino-acids were chosen. The two proteins had previously been designed as target proteins and exhibit distinctly different folds (FIG. 6 (A, B) target protein). The sequence of the amino acids for the target proteins during the design process was fixed while optimizing the sequence of the amino acids of the designed binding proteins. The predicted structures of the designed binding proteins exhibit high aligned confidence>0.81, good Rosetta binding energy<−58 REU and packing statistics>0.67 comparable to natural protein interfaces [48](FIG. 6 (A, B)). This indicates that designed binders form dimers with the target proteins. By visual inspection, it was noted that the binders designed for each target protein seem to all bind at the same interface indicating preferences for binding sites during the design process.

The MD simulations of the two designed binders for each of the two target proteins show unimodal distributions in the 2D CV space of global RMSF-total contacts, with mean RMSFs 2 Å and mean number of total contacts ranging from 300-400, consisting of significant numbers of individual intramonomer (100-200) and interfacial (40-120) contacts.

For the design of the binders, AF was provided with a template amino acid sequence for target proteins whose structure should remain fixed throughout the design process. The providing of templates enables the design of the binders without requiring MSA inputs for the target proteins. To reduce computation as much as possible, the amino acid sequence of the target proteins was cropped around the desired binding site. The cropped amino acid sequence was fed as a template into AF. Such templates keep computational costs fixed for the design of one or more of the binders that bind to the target protein, irrespective of a size of the target protein.

### Sequence Design Uncovers Signatures of Conformational Change in AlphaFold

To ascertain whether the AF sequence space contains information about protein conformational change when interacting with other proteins, the structures of two proteins known to have different monomeric and oligomeric states was predicted. These two structures are KaiB, a circadian clock protein [54], and amyloid-β involved in Alzheimer's disease [55].

As a monomer, KaiB adopts its ground-state structure, KaiBgs [54]. In complex with KaiC, KaiB changes conformation to the fold-switch stabilized state KaiBfs [54]. The AF prediction of KaiB as a monomer shows good agreement (RMSD 0.69 Å) with the native KaiBgs structure, while the prediction of KaiB in complex with KaiC shows good agreement with the native structure of KaiBfs (RMSD 1.92 Å). However, structural alignment of the predicted complex of KaiB and KaiC shows high RMSD (6.11 Å) to the structure of KaiBgs (FIG. 7 (A)), indicating that the AF prediction has captured a part of the conformational change between KaiBgs and KaiBfs.

In its monomeric state, the native amyloid-β forms an α-helical structure and changes conformation to a parallel β-sheet upon aggregation (FIG. 7 (A) grey) [105]. While not as accurate in terms of RMSD (>5 Å), AF captures the transition between the α-helical monomeric state and the parallel β-sheet oligomer well (Fig. (A) red). This indicates that indeed AF may have learned to predict conformational change upon protein binding in a limited way.

Furthermore, a set of oligomer designs exhibiting conformational change upon complex formation was identified (FIGS. 7 (B) and 12 Designed structures show a conformational change from an α-helix in the monomeric state (FIG. 7 (B), monomer structures) to a stack of parallel β-sheets characteristic for amyloids [55]. Both the monomeric and oligomeric states exhibit high predicted aligned confidence under AF (FIG. 7 (B) predicted error) and the oligomeric state shows binding energies<−88 REU and packing statistics>0.65 for all oligomers. This indicates that the complexes are stable under the Rosetta forcefield [86].

Similarly, the MD simulations show well-defined minima in the RMSF-contact space centering on low RMSFs (<2 Å) and a significant number of contacts (100-500). However, these conformation-switching open-ended oligomers do vary significantly compared to the other oligomers including conformation-retaining open-ended oligomers, as described previously. Whilst open-endedness is captured by a significant number of contacts in all-but-one sequential interfaces, the number of interfacial contacts (40-80) is either similar to or larger than the number of intramonomer contacts (10-60). This confirms the elongated conformational structure of monomers in the oligomeric state.

Heterodimers and homo-oligomers exhibiting conformational change upon complex formation were designed. By maximizing TM score between the monomeric and oligomeric state during optimization, it was possible to find proteins exhibiting the desired conformation change (FIG. 7 (C)). Interestingly, it was found that the resulting proteins show conformational changes beyond the amyloid-like transition found in the previous design experiments, indicating a larger variety of conformation changing proteins present in AF sequence space.

### Structure-to-Sequence Recovery

To generate improved sequences for structure candidates recovered from AF, a conditional autoregressive diffusion model (ADM) was trained on protein sequences and structures in the PDB. Conditional ADMs may learn to recover the amino acid sequence of a protein given the predicted structure of the protein, by optimizing the following loss:

$${L}_{ADM} ( θ ) = {𝔼}_{s , x ∼ {p}_{data} {s}^{′} ∼ {p}_{m a s k} ( {s}^{′} \vert s )} [ log {p}_{θ} ( s \vert {s}^{′} , x ) ]$$

where p_{θ} denotes the output distribution of protein sequences from the model, p_{data} denotes the data distribution, s denotes a protein sequence, x denotes a protein structure, and pₘₐₛₖ(s′|s) denotes a distribution of masked amino acid sequences s′ conditioned on a ground-truth sequence s. The distribution pₘₐₛₖ was chosen to be uniform over masked sequences. That is, the number of masked positions is sampled uniformly at random as are the masked positions. The distribution p_{θ}(s′|s, x) was parameterized as a product

Πᵢp_{θ}(sᵢ|s′,x)

of independent categorical distributions for each amino acid, as is customary for masked sequence modelling. The architecture of our ADM was based on the message-passing blocks of Protein MPNN (Protein Message Passing Neural Network). Our model consists of 6 MPNN (Message Passing Neural Network) blocks with 30 nearest neighbors, 128 node features and 128 edge features. Before passing the protein backbone to the MPNN, Gaussian noise was added with mean μ=0.0 and standard deviation σ=0.3 Å to all atom positions. In contrast to Protein MPNN, node features are initialized with the embedding of the masked amino acid sequence and the model is trained to directly predict independent probability distributions for each masked amino acid of the masked amino acid sequence. This makes it easier to implement complex dependencies between amino acids in homo-oligomers or repeat proteins during sampling. The training set consisted of structures from PDB with a resolution better than 3.5 Å, deposited before January 2021. Validation and test sets were drawn from structures in PDB, deposited before January 2022, with less than 10% sequence identity to the training set. The training set was clustered at 40% sequence identity. Structures were sampled from the training set with a probability inversely proportional to cluster size without replacement. Structures were accumulated into batches until adding an additional chain would increase the batch size beyond 10k amino acids. The model on batches padded to 10k amino acids. For simplicity of implementation, the loss was averaged over all amino acids in a batch, as opposed to averaging per sequence. The model was trained for 250k steps using Adam (Kingma and Ba, 2014) with hyperparameters β1=0.9 and β2=0.98 and linear warm-up to a final learning rate of 1e-3 over the first 10k steps.

To recover (or re-design) the sequence of a predicted structure, sampled, e.g., from AF and subject to constraints, a masked sequence for ADM inference was initialized. The sequence is masked at all positions where the initial sequence was not specified to be maintained (or fixed). E.g., for the case of protein binder design, the binder's sequence is completely masked while the target protein's sequence is maintained intact (or fixed). A random one of the masked positions of the binder's sequence is sampled. For this random position, a probability distribution computed, wherein the probability of forbidden amino acids is set to zero. The probability distribution is re-scaled by an inverse temperature of 10, and an amino acid is sampled for the random position. In case the sampled random position is associated with one or more sequence constraints, amino acids are sampled or set for those positions affected by the one or more sequence constraints. This process is repeated until no more masked amino acids remain. In this way, 100 sequences are sampled per predicted protein structure. The sampled sequences are ranked based on a likelihood for each sampled sequence under the model. For all subsequent steps in the method, the sampled sequence with the highest likelihood may be used.

To ensure that the redesigned (or recovered) amino acid sequences still fold as expected, structures are re-predicted for the redesigned (or recovered) amino acid sequences using AlphaFold with 12 recycling iterations. The mean pLDDT and pTM are computed for those re-predicted protein structures and discard amino acid sequences for which the product pLDDT pTM<0.5. The RMSD between the inially predicted and re-predicted protein structures as well as CamSol solubility scores (Sormanni et al, 2015) for the protein structures are computed. All remaining re-predicted protein structures are ranked according to pLDDT·pTM, RMSD, and the CamSol solubility scores. High ranking ones of the re-predicted protein structures are selected for experimental validation.

### RcaT Inhibitor Design

The structure of RcaT-Sen2 was predicted using ColabFold with 96 recycling steps and early stopping with a tolerance of 0.1 Å. Around a putative active site, 100 amino acid crops were extracted as templates for fixed-target design. AlphaDesign was run to generate predicted (or candidate) structures of 50 and 100 amino acids with fitness L_{binder}(X|RcaT-Sen2; active-site), 2 recycling steps, suboptimality 0.1, population size 10. Per predicted (candidate) protein structure, 100 amino acid sequences were generated using the autoregressive diffusion model, whilst keeping the target amino acid sequence fixed. The amino acid sequence with the highest likelihood was kept, whilst predicted (candidate) protein structures with 1/NΣᵢ^{N} log p_{θ}(sᵢ|x)<0.1 nats were discarded. Protein structures were re-predicted with AF at 12 recycling steps, and re-predicted (candidate) protein structures with pLDDT·pTM<0.5 were discarded. The solubility scores with CamSol were computed for the non-discarded re-predicted protein structures. From these re-predicted protein structures, those were discarded with the CamSol solubility score<1. The remaining re-predicted (candidate) protein structures were codon-optimized for expression in E. coli.

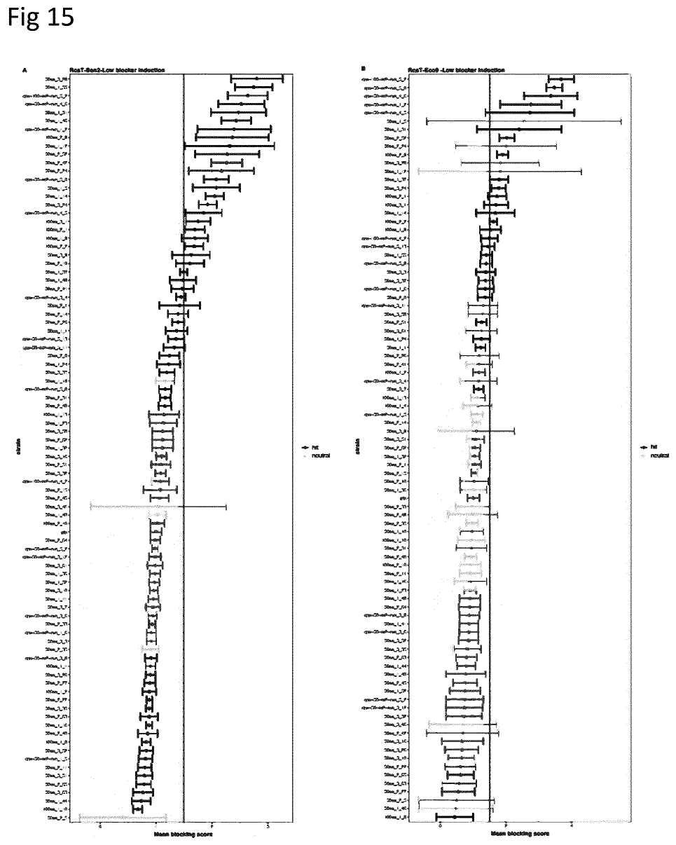

In a phenotypic experimental validation (see FIG. 15) of designed binders, a total of 88 proteins (of 50 or 100 amino acid length) were designed in silico to bind the active site and block the activity of the growth-inhibiting toxin RcaT-Sen2, part of the Retron-Sen2 toxin-antitoxin system (retron TA) [56]. Sixteen of them were also designed to additionally bind the active site of RcaT-Ecol, a divergent RcaT protein from E. coli (21.5% identical to RcaT-Sen2 on the protein level). To experimentally validate the function, the solubility, and/or the expressibility of the designed blockers, the ability of the designed blockers to inhibit the growth-inhibiting activity of RcaT-Sen2 was tested, by using a high-throughput reverse genetics screening approach called Toxin Inhibition Conjugation (TIC) [56]. In TIC, mobilizable gene-donor plasmids are en masse conjugated on agar plates with E. coli recipients transformed with a toxin plasmid. Normally, toxin-expressing strains do not grow, but growth is restored if the mobilizable plasmid encodes for a toxin blocker.

A gene-donor library was constructed by cloning each of the 88 designed blockers, designed according to the present disclosure, into an IPTG-inducible high-copy vector via Golden Gate assembly [57]. The gene-donor strains were arrayed in a 384-colony format on agar plates, conjugated them with E. coli BW25113 recipients transformed with an arabinose-inducible vector carrying RcaT-Sen2, and then tested the transconjugants for their ability to grow while co-expressing the RcaT-Sen2 toxin with the designed blockers.

To assess the promiscuity of the designed blockers, the gene-donor library was probed against RcaT-Eco9, a toxin 49% identical to RcaT-Sen2 from E. coli [56]. A quarter of the designed binders (22) restored bacterial growth in the presence of RcaT-Sen2 in varying degrees (FIG. 1A). Two of the top hits, RcaT-binder “cpx-50-nr2-run_5_0” and “50aa_1_55”, completely abolished RcaT-Sen2 toxicity and fully restored growth in the transconjugants. While primarily designed to target RcaT-Sen2, 15 constructs were also able to inhibit the toxicity of RcaT-Eco9 (FIG. 1B). Although the two toxins are not identical, their active sites are structurally similar, thus functional blockers likely bind both active sites (no blocker was found to only inhibit RcaT-Eco9). Notably, the top 5 hits of validated blockers for RcaT-Eco9 were designed to bind both RcaT-Sen2 and RcaT-Ecol active sites, potentially explaining their apparent promiscuity. Altogether, proteins designed according to the present disclosure can inhibit the activity of RcaT toxins in vivo, supporting the use of this tool for designing protein binders in silico.

### In Silico Protein Structure Prediction and Design Experiments

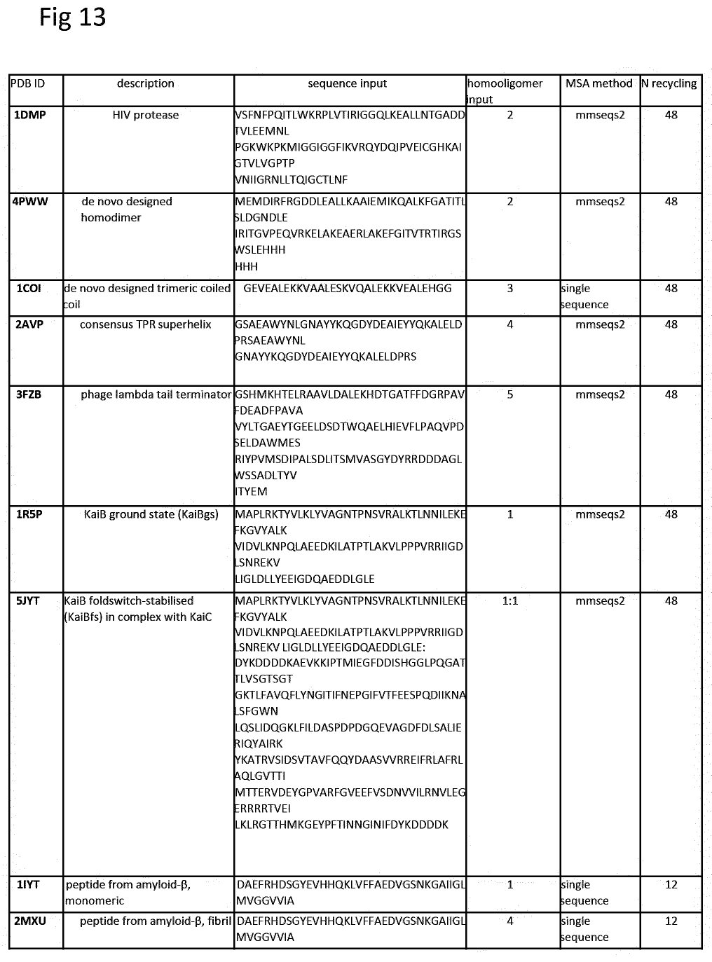

Complex formation and conformational change predictions were performed using Colabfold. Five AF-pretrained parameter sets (model_1_ptm, model_2_ptm, model_3_ptm, model_4_ptm, and model_5_ptm) were used for the predictions, and models were selected by highest predicted template matching score. Complex queries were predicted without paired MSA. The resulting top model was structurally aligned using PyMol for RMSD reporting. Parameters for prediction runs on proteins with PDB identifiers IR5P, 5JYT, IIYT, 2MXU are summarized in FIG. 13. For optimization, the evolutionary optimizer was used. Population size was set to 10. During mutation, population size was expanded by a factor of 2. Sequences with a suboptimality of at most 10% were considered for mutation and recombined. Recombination was applied by crossover with probability of 10% at each sequence position. Optimization was considered complete for sequences with L>1.5 for Lₘₒₙ, L_{cpx} and L>0.9 for ones of the fitness function including L_{cc} TABLE II and TABLE III (see above) contain further parameters of optimization runs performed. AF was configured for single-sequence use by disabling ensembling, templates, extra MSA features and restricting the number of MSA features to the number of monomers modelled. The number of AF iterations (recycling steps) was kept as a parameter for each optimization run (see TABLE II and TABLE III above). For larger protein complexes, the number of iterations was decreased to 2 to speed up computation. The AF-pretrained parameter set model_1_ptm (pre-trained using as large as possible a number of sequences in the MSA and structure templates referred to as model_1, and fine-tuned to predict the template matching score was used for all experiments. Finally, designed proteins of length 64 and larger were subdivided into a dictionary of fragments using Geometricus. Fragments were collected using the k-mer method with k=16 and the radius method with cutoff 10 Å. A collection of features representation was computed for all designed proteins and dimensionality was reduced using non-negative matrix factorization (NMF) with 50 components. Proteins were separated into 10 clusters using ward-linkage agglomerative clustering on their NMF components.

### CONCLUSION

A de novo protein design method based on optimization of the protein sequences of amino acids using evolutionary algorithms has been disclosed in this document. AlphaFold (AF) [4] has been embedded into the design loop as a prediction oracle. The optimization is facilitated by AF's output predicted confidence measures (or confidence metrics), namely the predicted local distance difference test (pLDDT) [39] and the predicted aligned error (pAE) [16]. A set of flexible fitness functions (or fitness function components, see TABLE I as well as the paragraphs preceding TABLE I) were designed that encode various design tasks aswell as an extendable platform for developing further ones of the fitness function for solving bespoke design problems. The method includes a step of post-processing the sequence of the designed proteins to be more native-like, as well as enhance solubility and improve expression when such proteins are synthesized experimentally. This has enabled a range of applications including de novo design of protein monomers, dimers, oligomers, context dependent conformational switchers and binders to target proteins.

The predicted protein structures are extensively validated using the Rosetta suite of protein design and structure prediction tools [21]. Fragment-assembly-based ab initio structure prediction [43] was used as an independent baseline for designed protein structures. In addition to this a further rigorous validation protocol was developed using all-atom molecular dynamics (MD) simulations that extends beyond conventionally used computational techniques for structure prediction evaluation. MD simulations enable extensive exploration of a putative native state; thus, instabilities are picked up as increases in global structural flexibility, loss of internal contacts within protein monomers and/or loss of interfacial contacts within complexes.

A subset of designed protein binders are also validated experimentally using an established phenotypic high-throughput reverse genetics screening approach called Toxin Inhibition Conjugation (TIC) [56]. For a set of 88 proteins designed to bind to the toxin protein RcaT-Sen2, 25% restore bacterial growth to some degree whilst 2 completely abolish Rcat-Sen2 activity. Several constructs even show broader abrogation of toxicity to a related but non-identical toxin (RcaT-Eco9). This demonstrates firstly that designed binders using this method are sufficiently soluble and expressible in vivo to be utilized in such experiments and furthermore, that the in silico design method results in notable binders capable of completely inhibiting the corresponding functionality of the target protein.

The method of this document has been applied to design de novo monomer proteins starting from completely random sequences of the amino acids ranging in size from—32-256 amino acids in length, based on the fitness function, in which pLDDT and pAE were combined. This method results in a range of structurally stable de novo designed monomer proteins with diverse folds. Using AF's functionality to predict the protein complexes, complex prediction on a number of systems showing good structural agreement was carried out. By further specifying the function based on globular compactness together with complex prediction, a range of stable de novo protein complexes can be designed including homodimers, heterodimers, and homo-oligomers from trimers to hexamers. Orthogonality between pairs of dimers can be shown, thus the method outlined in this document could have applicability in designing mutually exclusive combinations, for example, in protein logic gates [16]. Moreover, a number of open-ended predicted complexes have been observed, providing a potential route to the design of self-assembling systems [17].

A particularly intriguing subset of the predicted protein structures exhibit striking conformational changes between their monomeric and oligomeric state. Structural prediction of existing protein systems known to change conformation and/or fold between monomer and oligomer form, including amyloid—α-β switching, show that AF inherently contains signatures of conformational change. Conformation switching between monomer and oligomer forms was observed in some de novo designed open-ended oligomer systems. By defining the target function to maximize a structural difference between monomer and oligomer forms, the method can de novo design oligomers with this conformational switching property. Context dependent conformational switching is a desirable feature in synthetic biology applications. For example, designing proteins to self-assemble inside but not outside the cell may be achievable by design of membrane permeable α-helices that spontaneously switch into membrane impermeable β-sheeted filaments as accumulated intracellular concentration drives an equilibrium towards the oligomeric state.

The method enables the selection of the protein to be unmodified during the design loop—thus by combining this selection with a de novo designed protein starting from a random sequence of the amino acids, it is possible to design monomeric binders for the target protein, which may be pre-specified. Application of this approach to a chosen set of target proteins resulted in the design of stable binders that exhibit significant interfacial contacts across the same interface for a given target protein. The method may be further optimized towards therapeutic applications in potent biologic design.

### ACKNOWLEDGEMENTS

The inventors acknowledge support by the German Federal Ministry of Education and Research (BMBF) (de.NBI project: 031A537B) and the Volkswagen Foundation (contract 95826) as well as the Volkswagen Foundation “Experiment!Funding Initiative” (grant no. 93874-1). The inventors are grateful to Alessio Ling Jie Yang, Jacob Bobonis, Carlos Geert Pieter Voogdt and Athanasios Typas for carrying out the experimental validation study.

### REFERENCES

[1] Christine Zardecki, Chenghua Shao, Maria Voigt, and Stephen K. Burley. Protein data bank: 50 years of macromolecular structures enabling research and education. The FASEB Journal, 35, 2021.
[2] Brian Kuhlman and Philip Bradley. Advances in protein structure prediction and design. Nature Reviews Molecular Cell Biology, 20:681-697, 2019.
[3] Andriy Kryshtafovych, Torsten Schwede, Maya Topf, Krzysztof Fidelis, and John Moult. Critical assessment of methodsof protein structure prediction (ca-p)—round xiv. Proteins, 2021.
[4] John M Jumper, Richard Evans, Alexander Pritzel, Tim Green, Michael Figurnov, Olaf Ronneberger, Kathryn Tunyasuvu-nakool, Russ Bates, Augustin Zidek, Anna Potapenko, Alex Bridgland, Clemens Meyer, Simon A Kohl, Andy Ballard, Andrew Cowie, Bernardino Romera-Paredes, Stanislav Nikolov, Rishub Jain, Jonas Adler, Trevor Back, Stig Petersen, David A. Reiman, Ellen Clancy, Michal Zielinski, Martin Steinegger, Michalina Pacholska, Tamas Berghammer, Sebastian Bodenstein, David Silver, Oriol Vinyals, Andrew W. Senior, Koray Kavukcuoglu, Pushmeet Kohli, and Demis Hassabis. Highly accurate protein structure prediction with alphafold. Nature, 596:583-589, 2021.
[5] Kathryn Tunyasuvunakool, Jonas Adler, Zachary Wu, Tim Green, Michal Zielinski, Augustin Židek, Alex Bridgland, Andrew Cowie, Clemens Meyer, Agata Laydon, et al. Highly accurate protein structure prediction for the human proteome. Nature, 596(7873):590-596, 2021.
[6] Mehmet Akdel, Douglas Eduardo Valente Pires, Eduard Porta Pardo, Jürgen Jänes, Arthur O. Zalevsky, Bálint Mészáros, Patrick Bryant, Lydia L. Good, Roman A. Laskowski, Gabriele Pozzati, Aditi Shenoy, Wensi Zhu, Petras J. Kundrotas, Victoria Ruiz Serra, Carlos H M Rodrigues, Alistair S Dunham, David Burke, Neera Borkakoti, Sameer Velankar, Adam Frost, Kresten Lindorff-Larsen, Alfonso Valencia, Sergey Ovchinnikov, Janani Durairaj, David B. Ascher, Janet M Thornton, Norman E. Davey, Amelie Stein, Arne Elofsson, Tristan I. Croll, and Pedro Beltrão. A structural biology community assessment of alphafold 2 applications. bioRxiv, 2021.
[7] Milot Mirdita, Sergey Ovchinnikov, and Martin Steinegger. Colabfold-making protein folding accessible to all. bioRxiv, 2021.
[8] Ian R. Humphreys, Jimin Pei, Minkyung Baek, Aditya Krishnakumar, Ivan Anishchenko, Sergey Ovchinnikov, Jing Zhang, Travis J. Ness, Sudeep Banjade, Saket Bagde, Viktoriya G. Stancheva, Xiao-Han Li, Kaixian Liu, Zhi Zheng, Daniel J. Barrero, Upasana Roy, Israel S. Fernández, Barnabas Szakal, Dana Branzei, Eric C. Greene, Sue Biggins, Scott Keeney, Elizabeth A. Miller, J. Christopher Fromme, Tamara L. Hendrickson, Qian Cong, and David Baker. Structures of core eukaryotic protein complexes. bioRxiv, 2021.
[9] Richard Evans, Michael O'Neill, Alexander Pritzel, Natasha Antropova, Andrew Senior, Tim Green, Augustin Zidek, Russ Bates, Sam Blackwell, Jason Yim, Olaf Ronneberger, Sebastian Bodenstein, Michal Zielinski, Alex Bridgland, Anna Potapenko, Andrew Cowie, Kathryn Tunyasuvunakool, Rishub Jain, Ellen Clancy, Pushmeet Kohli, John M Jumper, and Demis Hassabis. Protein complex prediction with alphafold-multimer. bioRxiv, 2021.
[10] David D Boehr, Ruth Nussinov, and Peter E Wright. The role of dynamic conformational ensembles in biomolecular recognition. Nature chemical biology, 5(11):789-796, 2009.
[11] Subramanian Vivekanandan, Jeffrey R Brender, Shirley Y Lee, and Ayyalusamy Ramamoorthy. A partially folded structureof amyloid-beta (1-40) in an aqueous environment. Biochemical and biophysical research communications, 411(2):312-316, 2011.
[12] Kulkarni Madhurima, Bodhisatwa Nandi, and Ashok Sekhar. Metamorphic proteins: the janus proteins of structural biology. Open biology, 11(4):210012, 2021.
[13] S Kashif Sadiq, Frank Noè, and Gianni De Fabritiis. Kinetic characterization of the critical step in hiv-1 protease maturation. Proceedings of the National Academy of Sciences, 109(50):20449-20454, 2012.
[14] Neil J Bruce, Gaurav K Ganotra, Daria B Kokh, S Kashif Sadiq, and Rebecca C Wade. New approaches for computing ligand-receptor binding kinetics. Current opinion in structural biology, 49:1-10, 2018.
[15] Po-Ssu Huang, Scott E Boyken, and David Baker. The coming of age of de novo protein design. Nature, 537(7620):320-327, 2016.
[16] Zibo Chen, Ryan D Kibler, Andrew Hunt, Florian Busch, Jocelynn Pearl, Mengxuan Jia, Zachary L VanAernum, Basile I M Wicky, Galen Dods, Hanna Liao, et al. De novo design of protein logic gates. Science, 368(6486):78-84, 2020.
[17] Zibo Chen, Matthew C Johnson, Jiajun Chen, Matthew J Bick, Scott E Boyken, Baihan Lin, James J De Yoreo, Justin M Kollman, David Baker, and Frank DiMaio. Self-assembling 2d arrays with de novo protein building blocks. Journal of the American Chemical Society, 141(22):8891-8895, 2019.
[18] Aaron Chevalier, Daniel-Adriano Silva, Gabriel J Rocklin, Derrick R Hicks, Renan Vergara, Patience Murapa, Steffen M Bernard, Lu Zhang, Kwok-Ho Lam, Guorui Yao, et al. Massively parallel de novo protein design for targeted therapeutics. Nature, 550(7674):74-79, 2017.
[19] Michael J Dougherty and Frances H Arnold. Directed evolution: new parts and optimized function. Current opinion in biotechnology, 20(4):486-491, 2009.
[20] Xingjie Pan and Tanja Kortemme. Recent advances in de novo protein design: Principles, methods, and applications. Journal of Biological Chemistry, page 100558, 2021.
[21] Andrew Leaver-Fay, Michael D. Tyka, Steven M. Lewis, Oliver F. Lange, James M Thompson, Ron Jacak, Kristian Kaufman, Paul D. Renfrew, Colin A. Smith, William Sheffler, Ian W. Davis, Seth Cooper, Adrien Treuille, Daniel J. Mandell, Florian Richter, Yih-En Andrew Ban, Sarel Jacob Fleishman, Jacob E. Corn, David E. Kim, Sergey Lyskov, Monica Berrondo, Stuart G. Mentzer, Zoran Popovic, James J Havranek, John Karanicolas, Rhiju Das, Jens Meiler, Tanja Kortemme, Jeffrey J. Gray, Brian Kuhlman, David Baker, and Philip Bradley. Rosetta3: an object-oriented software suite for the simulation and design of macromolecules. Methods in enzymology, 487:545-74, 2011.
[22] Po-Ssu Huang, Yih-En Andrew Ban, Florian Richter, Ingemar Andre, Robert M. Vernon, William R. Schief, and David Baker. Rosetta remodel: A generalized framework for flexible backbone protein design. PLoS ONE, 6, 2011.
[23] Brian Kuhlman, Gautam Dantas, Gregory C. Ireton, Gabriele Varani, Barry L. Stoddard, and David Baker. Design of a novel globular protein fold with atomic-level accuracy. Science, 302:1364-1368, 2003.
[24] Bruno E. Correia, John T. Bates, Rebecca J. Loomis, Gretchen Baneyx, Chris Carrico, Joseph G. Jardine, Peter Rupert, Colin E. Correnti, Oleksandr Kalyuzhniy, Vinayak Vittal, Mary J. Connell, Eric Stevens, Alexandria Schroeter, Man Chen, Skye MacPherson, Andreia M. Serra, Yumiko Adachi, Margaret A. Holmes, Yuxing Li, Rachel E. Klevit, Barney S. Graham, Richard T. Wyatt, David Baker, Roland K. Strong, James E. Crowe, Philip R. Johnson, and William R. Schief. Proof of principle for epitope-focused vaccine design. Nature, 507:201-206, 2014.
[25] Yang Hsia, Jacob B. Bale, Shane Gonen, Dan Shi, William Sheffler, Kimberly K. Fong, Una Nattermann, Chunfu Xu, Po-Ssu Huang, Rashmi Ravichandran, Sue Yi, Trisha N. Davis, Tamir Gonen, Neil P. King, and David Baker. Design of ahyperstable 60-subunit protein icosahedron. Nature, 535:136-139, 2016.
[26] Florian Richter, Andrew Leaver-Fay, Sagar D. Khare, Sinisa Bjelic, and David Baker. De novo enzyme design using rosetta3. PLoS ONE, 6, 2011.
[27] Ali Madani, Bryan McCann, Nikhil Naik, Nitish Shirish Keskar, Namrata Anand, Raphael R. Eguchi, Po-Ssu Huang, and Richard Socher. Progen: Language modeling for protein generation. bioRxiv, 2020.
[28] Alex Hawkins-Hooker, Florence Depardieu, Sebastien Baur, Guillaume Couairon, Arthur Chen, and David Bikard. Generating functional protein variants with variational autoencoders. PLoS Computational Biology, 17, 2021.
[29] Donatas Repecka, Vykintas Jauniskis, Laurynas Karpus, Elzbieta Rembeza, Jan Zrimec, Simona Poviloniene, Irmantas Rokaitis, Audrius Laurynenas, Wissam Abuajwa, Otto Savolainen, Rolandas Meskys, Martin K. M. Engqvist, and Aleksej Zelezniak. Expanding functional protein sequence space using generative adversarial networks. bioRxiv, 2019.
[30] Namrata Anand and Possu Huang. Generative modeling for protein structures. In NeurIPS, 2018.
[31] Hao Huang, Boulbaba Ben Amor, Xichan Lin, Fan Zhu, and Yi Fang. G-vae, a geometric convolutional vae for protein structure generation. ArXiv, abs/2106.11920, 2021.
[32] Jingxue Wang, Huali Cao, John Zeng Hui Zhang, and Yifei Qi. Computational protein design with deep learning neural networks. Scientific Reports, 8, 2018.
[33] Alexey Strokach, David Becerra, Carles Corbi-Verge, Albert Perez-Riba, and Philip M. Kim. Fast and flexible protein design using deep graph neural networks. Cell systems, 2020.
[34] Ivan Anishchenko, Tamuka Martin Chidyausiku, Sergey Ovchinnikov, Samuel J Pellock, and David Baker. De novo protein design by deep network hallucination. bioRxiv, 2020.
[35] Christoffer H Norn, Basile I. M. Wicky, David Juergens, Sirui Liu, David E. Kim, Doug K Tischer, Brian Koepnick, Ivan V. Anishchenko, David Baker, and Sergey Ovchinnikov. Protein sequence design by conformational landscape optimization. Proceedings of the National Academy of Sciences of the United States of America, 118, 2021.
[36] Lewis Moffat, Joe G Greener, and David T Jones. Using alphafold for rapid and accurate fixed backbone protein design. bioRxiv, 2021.
[37] Ethan C. Alley, Grigory Khimulya, Surojit Biswas, Mohammed AlQuraishi, and George M. Church. Unified rational protein engineering with sequence-based deep representation learning. Nature Methods, pages 1-8, 2019.
[38] Jianyi Yang, Ivan V. Anishchenko, Hahnbeom Park, Zhenling Peng, Sergey Ovchinnikov, and David Baker. Improved protein structure prediction using predicted inter residue orientations. Proceedings of the National Academy of Sciences, 117:1496-1503, 2020.
[39] Valerio Mariani, Marco Biasini, Alessandro Barbato, and Torsten Schwede. lddt: a local superposition-free score for comparing protein structures and models using distance difference tests. Bioinformatics, 29:2722-2728, 2013.
[40] S. Sinai, Richard Wang, Alexander Whatley, Stewart Slocum, Elina Locane, and Eric D. Kelsic. Adalead: A simple and robust adaptive greedy search algorithm for sequence design. ArXiv, abs/2010.02141, 2020.
[41] Yang Zhang and Jeffrey Skolnick. Scoring function for automated assessment of protein structure template quality. Proteins: Structure, 57, 2004.
[42] Eric Jang, Shixiang Shane Gu, and Ben Poole. Categorical reparameterization with gumbel-softmax. ArXiv, abs/1611.01144, 2017.
[43] Kim T. Simons, Richard Bonneau, Ingo Ruczinski, and David Baker. Ab initio protein structure prediction of casp iii targets using rosetta. Proteins: Structure, 37, 1999.
[44] D. T. Jones. Protein secondary structure prediction based on position-specific scoring matrices. Journal of molecular biology, 292 2:195-202, 1999.
[45] Lewis Moffat and David T. Jones. A deep semi-supervised framework for accurate modelling of orphan sequences. bioRxiv, 2020.
[46] Rebecca F. Alford, Andrew Leaver-Fay, Jeliazko R. Jeliazkov, Matthew J. O'Meara, Frank Dimaio, Hahnbeom Park, Maxim V. Shapovalov, Paul D. Renfrew, Vikram Khipple Mulligan, Kalli Kappel, Jason W. Labonte, Michael S. Pacella, Richard Bonneau, Philip Bradley, Roland L. Dunbrack, Rhiju Das, David Baker, Brian Kuhlman, Tanja Kortemme, and Jeffrey J. Gray. The rosetta all-atom energy function for macromolecular modeling and design. Journal of chemical theory and computation, 13 6:3031-3048, 2017.
[47] Steven M. Lewis and Brian Kuhlman. Anchored design of protein-protein interfaces. PLoS ONE, 6, 2011.
[48] William Sheffler and David Baker. Rosettaholes: Rapid assessment of protein core packing for structure prediction, refinement, design, and validation. Protein Science, 18, 2009.
[49] James Maier, Carmenza Martinez, Koushik Kasavajhala, Lauren Wickstrom, Kevin Hauser, and Carlos Simmerling. ff14sb: Improving the accuracy of protein side chain and backbone parameters from ff99sb. Journal of chemical theory and computation, 11 8:3696-713, 2015.
[50] Ulrich Essmann, Lalith E. Perera, Max L. Berkowitz, Thomas A. Darden, Hsing-Chou Lee, and Lee G. Pedersen. A smooth particle mesh Ewald method. Journal of Chemical Physics, 103:8577-8593, 1995.
[51] Janani Durairaj, Mehmet Akdel, Dick de Ridder, and Aalt D. J. van Dijk. Geometricus represents protein structures as shape-mers derived from moment invariants. Bioinformatics, 36 Supplement_2: i718-i725, 2020.
[52] Daniel D. Lee and H. Sebastian Seung. Learning the parts of objects by non-negative matrix factorization. Nature, 401:788-791, 1999.
[53] Nicholas F. Polizzi, Yibing Wu, Thomas Lemmin, Alison M. Maxwell, Shao-Qing Zhang, Jeff Rawson, David N. Beratan, Michael J. Therien, and William F. DeGrado. De novo design of a hyperstable non-natural protein-ligand complex with sub-å accuracy. Nature chemistry, 9 12:1157-1164, 2017.
[54] Roger Tseng, Nicolette F Goularte, Archana G. Chavan, Jansen Luu, Susan E Cohen, Yong-Gang Chang, Joel Heisler, Sheng Li, Alicia K. Michael, Sarvind Tripathi, Susan S. Golden, Andy LiWang, and Carrie L. Partch. Structural basis of the day-night transition in a bacterial circadian clock. Science, 355:1174-1180, 2017.
[55] Rodrigo Gallardo, Neil A. Ranson, and Sheena E. Radford. Amyloid structures: much more than just a cross-β fold. Current opinion in structural biology, 60:7-16, 2019.
[56] Bobonis, J., Mitosch, K., Mateus, A., Karcher, N., Kritikos, G., Selkrig, J., Zietek, M., Monzon, V., Pfalz, B., Garcia-Santamarina, S., Galardini, M., Sueki, A., Kobayashi, C., Stein, F., Bateman, A., Zeller, G., Savitski, M. M., Elfenbein, J. R., Andrews-Polymenis, H. L., Typas, A., 2022. Bacterial retrons encode phage-defending tripartite toxin-antitoxin systems. Nature 1-7. https://doi.org/10.1038/s41586-022-05091-4
[57] Engler, C., Kandzia, R., Marillonnet, S., 2008. A One Pot, One Step, Precision Cloning Method with High Throughput Capability. PLoS ONE 3, e3647. https://doi.org/10.1371/journal.pone.0003647
[58] Kritikos, G., Banzhaf, M., Herrera-Dominguez, L., Koumoutsi, A., Wartel, M., Zietek, M., Typas, A., 2017. A tool named Iris for versatile high-throughput phenotyping in microorganisms. Nature Microbiology 2, 17014. https://doi.org/10.1038/nmicrobiol.2017.14

1. A computer implemented method for designing at least one protein comprising
creating at least one amino acid sequence to be tested, wherein ones of the amino acids, contained in the amino acid sequence to be tested, are selected according to a probability distribution;
predicting, from the at least one amino acid sequence, structural properties of the at least one protein;
calculating, based on the structural properties of the at least one protein, a value of a fitness function for the at least one amino acid sequence to be tested;
selecting or deselecting, dependent on the value of the fitness function, the at least one amino acid sequence to be tested.

2. The method of claim 1, further comprising altering the at least one amino acid sequence to be tested, by changing at least one of the amino acids contained in the amino acid sequence to be tested.

3. The method of claim 2, further comprising repeating the altering of the at least one amino acid sequence until the value of the fitness function exceeds a pre-selected threshold.

4. The method of claim 1, wherein the predicting of the structural properties of the least one protein, contained in the amino acid sequence to be tested, comprises predicting locations of the amino acids with respect to each other and/or predicting an all-atom structure of the at least one protein.

5. The method of claim 1, wherein the altering comprises mutating and/or recombining the at least one amino acid sequence to be tested.

6. The method of claim 1, wherein the structural properties are predicted using AlphaFold.

7. The method of claim 1, wherein the fitness function is defined according to biological and/or physicochemical properties of the at least one protein.

8. The method of claim 1, wherein the fitness function comprises one or more fitness function components, which represent the biological and/or physicochemical properties of the at least one protein.

9. The method of claim 1, further comprising inputting known structural properties associated with the at least one amino acid sequence or a target protein, to which the at least one protein is bindable, into AlphaFold as a structural template.

10. The method of claim 1, further comprising re-designing the selected at least one amino acid sequence to be tested, to make the re-designed at least one amino acid sequence more native-like and/or to enhance a solubility and/or an expressibility of the at least one protein.

11. The method of claim 10, further comprising re-predicting structural properties of the at least one protein based on the re-designed at least one amino acid sequence.

12. The method of claim 1, further comprising statistically recovering the at least one amino acid sequence to be tested, from the predicted structural properties of the least one protein.

13. The of claim 1, further comprising computationally validating the selected amino acid sequence using molecular dynamics and/or Rosetta ab-initio structure prediction.

14. The method of claim 1, further comprising experimentally validating the selected amino acid sequence by determining a solubility and/or an expressibility of the at least one protein.

15. The method of claim 1, wherein the at least one protein comprises a monomer, a homodimer, a heterodimer, a trimer, a tetramer, a pentamer, an oligomer, a protein complex, a component of a protein complex, a binder binding to a target protein, a protein exhibiting multiple conformations.
